(19) United States
(12) Patent Application Publication
(10) Pub. No.: US 2008/0293915 A1
(43) Pub. Date: Nov. 27, 2008

(54) Nanoscale Neuromodulating Platform For Retina Neuron Activation Apparatus and Method

(75) Inventor: David R. Pepperberg, Chicago, IL (US)

Correspondence Address: HUSCH BLACKWELL SANDERS LLP, 190 CARONDELET PLAZA, SUITE 600, ST. LOUIS, MO, 63105-3441, US

(73) Assignee: The Board of Trustees of the University of Illinois, Chicago, IL (US)

(21) Appl. No.: 11/912,754

(22) PCT Filed: Apr. 28, 2006
(86) PCT No.: PCT/US06/16232

§ 371 Date: Jun. 12, 2008

Related U.S. Application Data
(60) Provisional application No. 60/675,600, filed on Apr. 28, 2005.

(51) Int. Cl. C07K 2/00; C07C 245/06; C07D 261/12

(52) U.S. Cl. 530/300; 534/885; 548/243

(57) ABSTRACT

Postsynaptic membrance receptor proteins of retinal neurons proximal to the rods and cones mediate the transmission of visual signals at multiple types of chemical synapses in the normally functioning retina, and there is reason to believe that these proximal retinal neurons in certain cases remain functional despite the disease-induced loss of rod and cone visual signaling. The invention is a nanoscale molecular structure that can selectively attach to the extracellular face of specific membrane receptors of post-photoreceptor retinal neurons and, by modulating the postsynaptic receptor's activity in response to light, restore visual signaling in retina damaged by photoreceptor degenerative disease.

### CROSS-REFERENCE TO RELATED APPLICATIONS

This application claims priority from U.S. Provisional Patent Application No. 60/675,600 filed Apr. 28, 2005.

### STATEMENT REGARDING FEDERALLY SPONSORED RESEARCH

The present invention is supported by a R03 grant number EY13693 from the National Institutes of Health. The U.S. Government has certain rights in the invention.

### APPENDIX

Not Applicable.

### BACKGROUND OF THE INVENTION

1. Field of the Invention

The present invention relates to nanoscale neuromodulator platform apparatuses for opening in the presence of light the ion channels of receptors in postsynaptic neurons of damaged or diseased retinas.

2. Related Art

Photoreceptor disease and current therapeutic strategies: Retinal degenerative diseases such as age-related macular degeneration (ARMD) involve the progressive dysfunction and deterioration of rod and cone photoreceptors (e.g., Jackson et al., 2002). There is evidence that photoreceptor loss can lead directly or indirectly to diminished function of proximal, i.e., post-photoreceptor, retinal neurons (e.g., Strettoi et al., 2003). However, in certain cases these proximal neurons appear largely to retain their capacity for neural signaling (Medeiros & Curcio, 2001; Varela et al., 2003; Marc et al., 2003; Strettoi et al., 2003; Cuenca et al., 2004); the retina's loss of visual function follows from the inability of the deteriorating rods and cones to stimulate the postsynaptic membrane receptor proteins of post-photoreceptor neurons. Present research aimed at developing therapies for ARMD and related blinding diseases includes efforts based on photoreceptor rescue/replacement through genetic engineering, cell transplantation, and the provision of growth factors and protective biochemical agents (La Vail et al., 1998; Hauswirth & Lewin, 2000; Acland et al., 2001; Gouras & Tanabe, 2003; Wang et al., 2004). There is a need to achieve the restoration of visual function by a prosthetic device that electrically stimulates retinal neurons (Peachey & Chow, 1999; Humayun & de Juan, 1998; Rizzo et al., 2001; Zrenner, 2002; Margalit et al., 2002; Humayun et al., 2003) or focally delivers neurotransmitters within the retina (Iezzi et al., 2002; Gasperini et al., 2003; Peterman et al., 2003, 2004). Common to the current designs of retinal prostheses is a macroscopic structure (i.e., dimensions of ˜mm or greater) intended for implantation and interfacing with remaining healthy post-photoreceptor neurons. However, a major hurdle inherent in these approaches is the difficulty of achieving, with a macroscopic implanted device, the microlocalization and specificity of neuronal stimulation recognized as critical for the retina's spatial resolution of visual stimuli.

Normal photoreception: Visual signaling in rod and cone photoreceptors of the vertebrate retina begins with photoisomerization of the 11-cis retinal chromophore of visual pigment in the rod and cone outer segments. This photoisomerization event converts the retinal to all-trans form and initiates activating conformational changes of the protein (opsin) moiety of the pigment. Pigment photoactivation in turn initiates a chain of biochemical reactions that generate an electrical response. These activating stages of phototransduction, and reactions including those that deactivate the pigment and downstream transduction intermediates, determine the peak amplitude and time course of the electrical response to light (Burns & Baylor, 2001; Arshavsky et al., 2002). Complete recovery of the transduction machinery after illumination, i.e., complete dark adaptation of the photoreceptor, requires the action of metabolic and transport reactions that remove the all-trans retinal chromophore from opsin and provide resynthesized 11-cis retinal that binds to opsin, thereby regenerating photosensitive pigment (Saari, 2000; McBee et al., 2001). The photoreceptor electrical response transiently down-regulates the release of L-glutamate neurotransmitter at chemical synapses formed with retinal horizontal and bipolar cells. Resulting changes in the activity of postsynaptic membrane receptors of the bipolars produce a bipolar cell electrical response, thereby conveying visual signals initiated in the photoreceptors to neurons of the inner retina (Dowling, 1987; Wu & Maple, 1998; Thoreson & Witkovsky, 1999; Nawy, 2000).

There is a need to develop nanoscale molecular structures (“platforms”) that can selectively attach to the extracellular face of postsynaptic membrane receptor proteins in second-order neurons of the human retina, and, by modulating the receptor's activity in response to light, restore visual signaling in retina damaged by photoreceptor degenerative disease. There is a need for a platform expressing GABA_{C} receptors, a ligand-gated ion channel of retinal bipolar cells.

There is a need in the art to express and isolate the extracellular domain and full-length sequence of the GABA_{C} receptor. In vitro testing of platform components to be developed in the project will utilize isolated GABA_{C} protein as a model of the native receptor. Amino acid sequence and biochemical data from acetylcholine binding protein and ligand-gated ion channels of the GABA receptor family suggest that an N-terminal segment (˜250 amino acids in length) of the GABA_{C} ρ1 subunit forms the primary extracellular domain of the native GABA_{C} receptor, and that pentameric complexes of this segment contain the GABA-binding sites of the native receptor. To characterize the interactions of platform components specifically with the target receptor protein, we will use bacterial and baculovirus expression systems, and protein isolation and solubilization techniques, to prepare functional GABA_{C} extracellular domain as well as solubilized/membrane-associated full-length sequence.

There is a need in the art to synthesize/characterize tetherable GABA analogs that exhibit agonist/antagonist activity at the GABA_{C} ligand-binding site. The effector component of the envisioned molecular platform, i.e., the component directly interacting with the receptor's GABA-binding site, will consist of a GABA analog (agonist or antagonist) covalently incorporated into the platform through a molecular photoswitch and linker. Recent data indicate that a biotin-terminated, N-acyl derivative of the GABA_{C} agonist muscimol exhibits activity at GABA_{C} receptors expressed in Xenopus oocytes. Related derivatives of muscimol as well as similarly derivatized phosphinic acid analogs of GABA should be developed. In electrophysiological, in situ binding and in vitro reconstitution experiments, we will test the GABA_{C} activities of candidate effectors joined to a first-generation (azobenzene) photoswitch and linker. Primary preparations to be used for electrophysiological testing will be GABA_{C}-expressing Xenopus oocytes and mammalian cell lines, and GABA_{C} receptors of isolated retinal bipolar cells.

There is a need in the art to achieve anchoring of the platform to the GABA_{C} receptor. Operation of the platform will require its covalent binding to GABA_{C} at a defined site on the extracellular domain. Phage display technology may identify a suitable molecular anchor. Using both isolated target protein (extracellular domain/full-length sequence) and whole cells expressing GABA_{C}, a 12-mer peptides is selected that exhibits high-affinity noncovalent binding to the GABA_{C} extracellular domain. Receptor engineering (cysteine substitution, to test the binding activity of peptides derivatized with a thiol-reactive agent), photoaffinity derivatization of the peptide, computational modeling, and biophysical/electrophysiological testing, may optimize the sequence of the peptide and map its site of interaction with GABA_{C}. A “filtered” set of candidate peptides may identify photoaffinity-derivatized peptides that exhibit “silent” covalent binding to GABA_{C}, i.e., covalent attachment that does not perturb receptor electrophysiology.

There is a need in the art to achieve photic control of GABA_{C} activity. The central objective is to achieve light-dependent regulation of GABA_{C} functional properties in one or more model cell systems, by a platform that consists of an effector/photoswitch/linker assembly coupled to an anchor, and that binds covalently to the native receptor in site-specific and silent fashion. There is a need to synthesize/test second-generation photoswitches, e.g., “push-pull” azobenzene derivatives, the operation of which in the assembled platform will afford sensitivity to visible light, and will yield relaxation times and other platform kinetic properties suitable for physiological regulation of the receptor.

### SUMMARY OF THE INVENTION

The essential role of rod and cone photoreceptors is to generate transient light-dependent molecular signals (reduced glutamate release) that modulate the activities of postsynaptic membrane receptors of retinal bipolar and horizontal cells. Thus, the loss of retinal function resulting from photoreceptor degeneration could in principle be circumvented by introducing, at the postsynaptic membrane of proximal retinal neurons, molecular structures that could bind to the membrane receptors and modulate receptor activity in light-dependent fashion. The broad requirements of such a structure would include: accessibility to the receptor protein (i.e., dimensions ˜nm to allow diffusion to the receptor when introduced into the retinal extracellular milieu); specificity of attachment to the extracellular face of the target receptor protein; high photic sensitivity (high absorptivity of light incident on the retina); ability to generate sufficiently large and long-lived changes in receptor activity upon photon absorption; spontaneous shut-off and recovery to the pre-illumination state following light absorption; biological compatibility (non-toxicity); and long-term physical/chemical stability, including resistance to native degradative enzymes.

FIG. 1 illustrates signal transmission at a normally functioning chemical synapse for which the postsynaptic membrane receptor is a hypothetical ligand-gated ion channel (LGIC) consisting of two subunits and a single ligand-binding site. Here, neurotransmitter (filled circles) released from the presynaptic neuron in response to stimulation diffuses across the synaptic cleft and binds to the postsynaptic membrane receptors. The resulting activation of these receptor proteins opens transmembrane ion channels (inward-pointing arrow), thereby generating an electrical signal in the postsynaptic neuron. FIG. 2 describes the function of a representative, ultimately envisioned NNP under disease conditions where the pre-synaptic neuron has deteriorated. The NNP consists of derivatized native neurotransmitter or analog (small filled circle), here termed an effector, tethered to a structure (open circle labeled NNP) that incorporates a photoswitch, and an anchoring component (open triangle) that selectively and covalently attaches the NNP to the extracellular face of the receptor protein. Photon absorption by the NNP produces a transient conformational change in a linker arm that moves the effector to the receptor protein's ligand-binding site and thereby transiently activates the receptor, i.e., opens the receptor's ion channel. As a self-contained photosensor (i.e., not dependent on interfacing with a macroscopic structure) with localized stimulating activity, the envisioned NNP would achieve the critical feature of microspecific functionality.

FIG. 2 illustrates a “nanoscale neuromodulating platform (NNP)” of the present invention.

In FIG. 2 NNPs introduced as a suspension into the vicinity of the retina (intravitreal or subretinal injection into the eye) would diffuse through extracellular clefts to target membrane receptors, where high-affinity binding to the receptor's extracellular face would anchor the NNP. Illustrated molecular structures are not shown to scale.

Molecular structures (NNPs) will selectively bind to GABA_{C} postsynaptic receptors and render the receptor's channel gating activity controllable by light. End products of an iterative approach (FIG. 3) will be optimized separate/coupled platform components and configurations that may be maintained for incorporation within the ultimate, fully functional platform. A given system under study may consist of a ligand/platform preparation (e.g., a ligand such as untethered candidate effector or phage-derived peptide anchor; test platform such as an effector-photoswitch-anchor conjugate) and a target protein preparation (e.g., GABA_{C}-expressing oocyte or isolated GABA_{C} extracellular domain). This system will involve determining the interactions between the ligand and target under defined conditions. In vitro reconstitution procedures may determine the strength and specificity with which the ligand or platform binds to the target. Cell-based binding assays involving the incubation of GABA_{C}-expressing cells with test ligand/platform may quantify the strength/specificity of binding to GABA_{C} in situ. Here, using model and native GABA_{C}-expressing cells (oocytes, mammalian cell line, and isolated retinal bipolar cells) and, subsequently, intact retina (isolated retina and intact eye), may be used for electrophysiological determination of ligand/platform activity of the test preparation in GABA_{C}-mediated ion channel gating.

Focus on GABA_{C} receptors: The developments of NNPs will employ GABA_{C} receptors as a model postsynaptic receptor protein. The GABA_{C} receptor is a member of the ligand-gated ion channel superfamily, which includes nicotinic acetylcholine receptors as well as GABA_{A}, glycine and 5-HT₃ receptors. Functional receptors of this family consist of five subunits, with each protein subunit consisting of a large extracellular N-terminal domain, four transmembrane segments connected by a small extracellular domain, and both a small and a large intracellular domain. The subunit's C-terminal domain is predicted to be extracellular and to contain only a few amino acids (Betz, 1990; Qian & Ripps, 2001), and we shall henceforth refer to the GABA_{C} N-terminal extracellular domain as “the extracellular domain”. GABA receptors are widely distributed in CNS tissue, including retina. GABA_{C} receptors are present on all subtypes of bipolar cells in the retina, with locations including both proximal and distal regions of these cells (Qian & Dowling, 1994; Enz et al., 1996; Qian et al., 1997; Lukasiewicz & Shields, 1998; Euler & Wassle, 1998). GABA_{C} receptors are, by comparison with GABA_{A} receptors, non-desensitizing and exhibit slow response kinetics (Feigenspan et al., 1993; Qian & Dowling, 1993; Pan & Lipton, 1995). GABA_{C} receptor activities are an integral part of retinal function, and GABA_{C}-mediated activity is specifically detectable in electroretinographic (ERG) recordings obtained from the intact eye (McCall et al., 2002; Dong & Hare, 2002).

Some prior art argued that metabotropic and ionotropic glutamate receptors (mGluR6 and AMPA glutamate receptors), the native postsynaptic membrane receptors at rod and cone synapses with ON and OFF bipolar cells, are the preferred targets of investigation in a project aimed at bypassing the rod and cone photoreceptors. However, recent studies indicate significant down-regulation of glutamate receptors on bipolar cells of degenerated retina (Varela et al., 2003; Strettoi et al., 2003; Cuenca et al., 2004). In addition, by contrast with the case of multiple glutamate receptors, ON and OFF bipolars possess the same types of GABA receptors (Euler & Wassle, 1998; Shields et al., 2000). Thus, tetherable effectors identified in the present project could ultimately have application in NNPs designed for both ON and OFF bipolars. A second advantage of GABA_{C} receptors concerns the size of the receptor-mediated electrical response. By contrast with the relatively small size of desensitized responses mediated by mGluR6 and AMPA glutamate receptors, and despite the small single-channel conductance of GABA_{C} receptors, overall (i.e., population-summed) GABA_{C}-mediated responses of bipolar cells are relatively large, do not desensitize, and are readily measured in mechanically/enzymatically isolated retinal bipolars (Feigenspan et al., 1993; Gillette & Dacheux, 1995; Qian & Dowling, 1995; Qian et al., 1997). The known pharmacology of GABA_{C} receptors is not as extensive as that for GABA_{A} receptors (Johnston, 1996). However, a further advantage of the GABA_{C} receptor, one especially relevant to the present project's use of receptor expression in model cells (oocytes and mammalian cell lines), is the relatively limited diversity of GABA_{C} receptor subunits in retinal neurons. For example, only three GABA_{C} subunits (ρ1, ρ2 and ρ3) are expressed in rat retina, and only two of these are expressed in bipolar cells (ρ1 and ρ2) (Enz et al., 1995, 1996; Ogurusu & Shingai, 1996). By contrast, 15 GABA_{A} subunits have been cloned from CNS neurons (Whiting et al., 1995; Mehta & Ticku, 1999), and most of these are expressed in retina (Wassle et al., 1998). Moreover, there is abundant evidence that the GABA_{C} ρ1 subunit readily associates to form functional homomeric receptors (Cutting et al., 1991; Zhang et al., 1995; Qian et al., 1998). The relative uniformity of native retinal GABA_{C} receptors, the workability of recording GABA_{C}-mediated responses in isolated bipolar cells, and the demonstrated functionality of GABA_{C} subunits in the simplest (i.e., homomeric) model system are of major advantage in developing molecular structures to interface with postsynaptic membrane receptors. Furthermore, GABA_{C} receptors share high homology with other LGICs, providing a foundation for extension of the technology to be developed to other LGICs such as the GABA_{A} receptor.

Express/isolate GABA_{C} extracellular domain and full length sequences are expressed and isolated. NNP development will involve the in vitro testing of candidate components with a model target receptor, the expressed (N-terminal) GABA_{C} extracellular domain. Many membrane proteins contain domains that, when expressed as isolated fragments, retain properties that mimic those of the native protein (e.g., Grauschopf et al., 2000). For example, Chen & Gouaux (1997) expressed linked extracellular domains of the native AMPA glutamate receptor and found that these domains exhibit glutamate-binding activity. Furthermore, an expressed portion of the GABA_{A} extracellular domain exhibits a benzodiazepine-binding property resembling that of the native receptor (Shi et al., 2003). In addition, acetylcholine binding protein (AchBP), a soluble binding protein of snail glia that exhibits significant sequence homology with GABA_{C} receptors and from which a crystal structure has recently been obtained (Brejc et al., 2001; Smit et al., 2001; Cromer et al., 2002), exists as a pentameric complex. These findings suggest that expressed GABA_{C} extracellular domain will exhibit folding, pentamer-forming and GABA-binding properties resembling those of native GABA_{C} receptors.

Both bacterial and baculovirus (sf9 cells) expression systems are used for preparation of the GABA_{C} extracellular domain. These two systems have complementary strengths. The bacterial system is a widely used system capable of yielding large amounts of expressed protein and has been used, in particular, to obtain a soluble N-terminal domain preparation of the AMPA glutamate receptor (Chen & Gouaux, 1997). The baculovirus system (baculovirus transfection of insect cells), which has been used to express both soluble and membrane proteins (e.g., Stauffer et al., 1991; Griffiths & Page, 1997; Hu & Kaplan, 2000; Gatto et al., 2001; Eisses & Kaplan, 2002; Massotte, 2003), also has distinct advantages. The insect cells are eukaryotic and can readily express mammalian proteins; the proteins are post-translationally processed appropriately (although there may be incomplete glycosylation); and cell culture of these cells is straightforward and relatively inexpensive. A specific advantage of the baculovirus system is its capacity to generate functional, multimeric membrane proteins. It is one of the most widely used systems for expressing these multimeric proteins because, unlike the bacterial system, the subunits of these proteins oligomerize well in this system (e.g., Eisses & Kaplan, 2002; Laughery et al., 2003). In addition, by contrast with mammalian cells, the baculovirus system is capable of high levels of expression of membrane proteins, a factor important for purified protein in multiple biophysical and biochemical assays. The proposed experiments to obtain full-length GABA_{C} will employ the baculovirus system; those to obtain GABA_{C} extracellular domain will employ the system (bacterial or baculovirus) that we find overall to be the more efficient with respect to solubility, purity and functionality of the expressed protein.

Tetherable GABA_{C} effectors are engineered. Receptor activation by the NNP is mediated by a tethered effector that in light-dependent fashion interacts with the GABA_{C} ligand-binding site. Tetherable GABA analogs can serve this function in the fully assembled platform. The known pharmacology of GABA_{C} receptors includes studies of muscimol (a potent agonist), and of phosphinic acid analogs that contain a (derivatizable) phosphorus atom in place of GABA's carboxylcarbon atom (Murata et al., 1996; Chebib et al., 1997a,b; Chebib & Johnston, 2000; Zhang et al., 2001; Johnston, 2002; Krehan et al., 2003). Of particular relevance are recent reports that indicate GABA receptor-binding activity by amide-linked GABA analogs, i.e., N-substituted forms that, unlike GABA, lack a protonatable nitrogen and are thus non-zwitterionic at neutral pH (Wang et al., 2000; Meissner & Haberlein, 2003). In addition, a GABA analog containing a similar N-amide linkage is recognized by GABA receptors of brain tissue (Carlier et al., 2002). Applicant's findings show that amide-linked, aminocaproyl-chain-containing derivatives of muscimol exhibit electrophysiological activity in GABA_{C}-expressing Xenopus oocytes (Vu et al., 2005; section C.2). Derivatized forms of muscimol, and phosphinic acid GABA analogs are synthesized to determine the activities of these compounds in electrophysiological and in vitro/in situ binding experiments. Two strategies involve conjugation of the test effector with azobenzene, a molecular photoswitch that here is employed as a first-generation photoswitch moiety. In both strategies, effector/photoswitch couples will be joined to a linear poly(ethylene glycol) (PEG) linker that in the fully assembled NNP will connect the effector/photoswitch to an anchoring component, and both strategies will involve biophysical/electrophysiological testing of effector/photoswitch/linker assemblies to identify effectors that meet projected, quantitative performance criteria. The main factor distinguishing the two strategies will be the length of the PEG linker (“long” vs. “short” chain), a feature anticipated to be key in governing the ultimate physiological performance of the effector at the GABA_{C} ligand-binding site. Azobenzenes have been widely used to light-regulate the properties of polymers and peptides, enzymes, and ionophores in vitro (Erlanger, 1976; Liu et al., 1997; Willner & Rubin, 1996; Pieroni et al., 1998; Borisenko et al., 2000; Dugave & Demange, 2003; Burns et al., 2004). The extensive use of azobenzenes as derivatizable photoswitches is based on their ease of synthesis as well as their physical and photochemical stability. The more stable trans isomer and the metastable cis isomer can be interconverted rapidly, efficiently and reversibly by light because they have distinct absorption maxima. Typically, irradiation in the near-UV (˜370 nm) produces 80-90% cis, and irradiation in the visible (>450 nm) yields 90% trans.

Platforms at the GABA_{C} extracellular face are selectively anchored. Microspecific functionality of the ultimately envisioned NNP will depend on its covalent attachment to the GABA_{C} extracellular face at a defined site distinct from the receptor's ligand-binding site. As the anchoring component to be joined with the effector/photoswitch/linker in the fully assembled NNP, we will identify 12-mer peptides that exhibit high-affinity noncovalent binding to the GABA_{C} extracellular domain, and that can be derivatized with a photoaffinity probe to afford covalent attachment. Phage display (Rodi et al., 2002) may be used to select the sequence(s) of the desired high-affinity peptide(s), a high-throughput, relatively low-cost technology (relative to generating monoclonal antibodies) that has been widely used to identify peptides with high affinity for specific molecular targets including transmembrane and soluble proteins (Sarrias et al., 1999; Whaley et al., 2000; Zurita et al., 2003). In the first of these, phage-displayed combinatorial peptide libraries may be screened against both whole-cell-expressed target receptor (cf. Goodson et al., 1994; Fong et al., 1994; Watters et al., 1997; Brown, 2000; Popkov et al., 2004) and the isolated, biotinylated (and immobilized) extracellular domain of the target (cf. Smith & Scott, 1993; Karatan et al., 2004; Scholle et al., 2004). Synthesized peptides of the sequences determined in this phage screening are tested for GABA_{C} binding activity in biophysical and electrophysiological procedures, to identify “first-generation” peptide ligands for further investigation. The second phase will employ combined biochemical, receptor engineering (cysteine substitution) and computational modeling approaches, together with biophysical/electrophysiological testing of candidate peptide ligands, to guide modification of the first-generation ligands and yield peptides whose sequences are optimized for high-affinity GABA_{C} binding; and to determine the GABA_{C} sites of peptide binding through photoaffinity derivatization of the peptide and analysis of the products of this covalent attachment reaction. The third phase will also involve peptide derivatization with a photoaffinity probe with the more stringent (than the second-phase research) objective of identifying, for native GABA_{C}, modes and sites of covalent attachment that preserve normal GABA_{C} function (“silent” attachment) and thus are suitable for anchoring the fully assembled NNP.

Photic control of GABA_{C} receptor activity is achieved. Simple azobenzenes, the first-generation photoswitch have the limitations of requiring UV light for activation and displaying slow thermal relaxation (time scale of hours or more). The latter property is extremely useful for prototype development and characterization. However, NNP functionality will require the photoswitch's spontaneous relaxation with kinetics compatible with GABA_{C} receptor physiology (time scale of seconds or less), as well as sensitivity to light in the visible range. Second-generation photoswitch compounds that address these limitations are synthesized and tested. One embodiment may be to construct derivatives of azobenzene possessing a red-shifted absorbance spectrum relative to simple azobenzenes (i.e., a λₘₐₓ in the visible range) and thermal relaxation on the desired (second- or sub-second-) time scale following photoisomerization. A prime justification for directing attention to azobenzene-based structures (push-pull azobenzenes and imines) is their successful application to the control of transmembrane ion channels. Of particular relevance to this embodiment is the demonstration, by Lester and colleagues, that both a freely-diffusing azobenzene analog of acetylcholine (Ach), and a closely related, receptor-tethered analog, afford light-dependent activation of nicotinic Ach receptors (Bartels et al., 1971; Lester & Nerbonne, 1982; Lester et al., 1986; Gurney & Lester, 1987). Further encouragement for the development of azobenzene-based, receptor-anchored effectors comes from a recent ground-breaking study by Banghart et al. (2004), who demonstrated light-regulated control of hippocampal cell-expressed K⁺ channels by a structure tethered to a (genetically engineered) cysteine on the protein, and linked via an azobenzene to a tetraethylammonium blocker of channel activity. However, both the system studied by Lester and co-workers, and that studied by Banghart et al. (2004) employed simple azobenzenes, and therefore required photic regeneration of the baseline (i.e., dark-adapted) state by light of a wavelength different from the activating wavelength. The use of the simple, slowly relaxing azobenzene structures (conjugation of an azobenzene-based photoswitch with an effector and linker), and the substantial body of literature describing the influence of substituents on the thermal and photochemical properties of azobenzene derivatives (e.g., Schanze et al., 1983; Asano & Okada, 1984; Kobayashi et al., 1987; Wachtveitl et al., 1997) is beneficial.

Pilot work was to develop a prototype system consisting of a macroscopic surface (dimensions ˜mm) coated with a redox-sensitive, chain-derivatized GABA analog and interfaced with a HgCdTe-based avalanche photodetector, and to use this system to test the feasibility of light-dependent activation of GABA_{C} receptors expressed in Xenopus oocytes. Milestones achieved in the R03-supported work included completion of a study of immobilized GABA analog (Saifuddin et al., 2003) and of the synthesis/testing of muscimol-biotin, a candidate tetherable GABA_{C} effectors (Nehilla et al., 2004; Vu et al., 2005).

Synthesis, immobilization and biophysical characterization of chain-derivatized analogs of GABA and muscimol. One embodiment will involve atomic force microscopy (AFM) testing of GABA_{C} extracellular domain and prototype NNP components tethered to a solid support. Using commercially obtained anti-GABA antibody as a model GABA-binding protein showed surface properties of a candidate chain-derivatized GABA analog. The analog consisted of a GABA moiety N-linked to biotin through an ethylene oxide chain. In AFM experiments employing surfaces coated with avidin-tethered biotinylated GABA analog and control surfaces lacking the analog, we found that incubation with anti-GABA antibody (employed here as a model GABA-binding protein) produced changes in surface topology, indicating interaction of the antibody with the analog's GABA moiety. The results obtained from this elementary model system provide evidence that tethering of a chain-derivatized GABA analog can preserve GABA-like biofunctionality. In another recently published study (Nehilla et al., 2004), assembled and characterized silicon platforms containing a chain-derivatized form of the GABA_{C} receptor agonist muscimol that may be used in this embodiment.

Electrophysiological activity of chain-derivatized muscimol is to identify tetherable analogs of GABA that exhibit agonist or antagonist activity at GABA_{C} receptors expressed in Xenopus oocytes and mammalian cells. The biotinylated GABA compound exhibited little if any electrophysiological activity in GABA_{C}-expressing oocyte. However, we have found that a biotinylated analog of the known GABA receptor agonist muscimol, henceforth termed muscimol-biotin (FIG. 4), exhibits significant activity (Vu et al., 2005). Synthesis of muscimol-biotin: Briefly, biotinamidocaproic acid N-hydroxysuccinimide ester was reacted with muscimol in N-methylpyrrolidinone in the presence of diisopropylethylamine. The product was purified to homogeneity by reversed-phase HPLC. Peaks were detected by absorbance at 210 nm (FIG. 5), collected, and lyophilized to afford muscimol-biotin. The muscimol-biotin product was judged to be 97% pure by ¹H NMR spectroscopy, with no detectable contamination of the HPLC-purified product peak by muscimol (limit of detection: ca. 1%). Muscimol-biotin was dissolved in DMSO, stored at 3° C., and diluted to desired concentrations in frog Ringer before testing on the oocyte. Electrophysiology: Procedures used for Xenopus oocyte preparation, including GABA_{C} expression, followed those described previously (Qian et al., 1998). Membrane currents were recorded from GABA_{C}-expressing oocytes by 2-electrode voltage clamp in a recently constructed (R03/IRIB-supported) apparatus. FIGS. 6-7 show results obtained for muscimol-biotin in GABA_{C}- and GABA_{A}-expressing oocytes. At GABA_{C} receptors (FIG. 6), muscimol-biotin exhibited agonist activity with an EC₅₀ of 20 μM and Hill coefficient of 4.4 (see legend), and this activity was suppressible by TPMPA, a known GABA_{C} antagonist. Muscimol-biotin also exhibited agonist activity at GABA_{A} receptors (FIG. 7), and this activity was suppressible by the known antagonist bicuculline. The finding of a Hill coefficient of 4.4 for GABA_{C} receptors specifically suggests a high cooperativity in GABA_{C} activation by muscimol-biotin; this cooperativity might reflect, for example, hydrophobic interactions among the alkyl chains of muscimol-biotin molecules at the GABA_{C} receptor.

FIG. 4 depicts structures of GABA, muscimol and muscimol-biotin. FIG. 5 depicts HPLC isolation of muscimol-biotin from a preparative reaction mixture: Waters Delta-Pak C₁₈ column (25×100 mm); elution with a linear gradient of 0-40% acetonitrile (0.08% TFA) in water (0.1% TFA) over 25 min. The three resolved peaks are N-hydroxysuccinimide and unreacted muscimol (1t), N-methylpyrrolidinone (2) and muscimol-biotin (3).

FIGS. 6 and 7 show the effects of muscimol-biotin on GABA_{C}- and GABA_{A}-expressing Xenopus oocytes. Left (FIG. 4): GABA_{C} receptors. (A) Response to 10 μM muscimol and 500 μM muscimol-biotin recorded from a single oocyte. (B) Response of a single oocyte to 50 μM muscimol-biotin and to the co-application of 50 μM muscimol-biotin and 200 μM TPMPA. (C) Responses recorded from a single oocyte on the presentation of varying concentrations (in μM) of muscimol (upper) and muscimol-biotin (lower). (D) Normalized peak amplitudes (mean ±SEM) of responses to muscimol and muscimol-biotin recorded from GABA_{C}-expressing oocytes (n=5 for muscimol; n=6 for muscimol-biotin). Here and in the right-hand panel D, peak amplitudes of all responses obtained from a given oocyte are normalized to the peak amplitude of the saturating response to muscimol; and fitted curves plot the Hill equation, r/rₘₐₓ=cⁿ/(cⁿ+EC₅₀ⁿ), where r/rₘₐₓ is the normalized response amplitude, c is the concentration of test substance, and n and EC₅₀ are fitted parameters. The fits yield EC₅₀=2.0 μM and n=1.2 for muscimol (open circles); and EC₅₀=20 μM and n=4.4 for muscimol-biotin (filled circles). Right (FIG. 7): GABA_{A} receptors. (A) Responses of a single oocyte to 100 μM muscimol-biotin and 10 μM GABA. (B) Responses of another oocyte to 2.5 μM muscimol-biotin alone, and to co-application of 2.5 μM muscimol-biotin and 100 μM bicuculline. (C) Family of responses to varying concentrations of muscimol-biotin and to a single, saturating concentration of muscimol (200 μM, thick trace) recorded from a single oocyte. (D) Normalized peak amplitudes (mean ±SEM) of responses recorded from GABA_{A}-expressing oocytes upon the application of muscimol (open circles) and muscimol-biotin (filled circles) (n=9). The fitted Hill equation curves yield n=0.74 and EC₅₀=4.8 μM for muscimol; and n=1.4 and EC₅₀=385 μM for muscimol-biotin.

FIG. 8 graphs whole-cell patch recording of GABA-elicited response of a neuroblastoma cell expressing the human GABA_{C} ρ1 subunit. Horizontal line: period of application of 10 μM GABA. FIG. 9 graphs (³H)GABA competition binding data obtained from GABA_{C}-expressing neuroblastoma cells. Data points are averages of duplicate samples. Result obtained in the absence of unlabeled GABA (B/B₀=100%) is arbitrarily positioned at log[GABA]=−9.3. The illustrated smooth curve was fitted to the data using Prism Graphpad software.

Electrophysiological and GABA-binding properties of GABA_{C}-expressing mammalian cells involve cell-based and in vitro reconstitution of test ligand binding to GABA_{C} receptors. In one embodiment, neuroblastoma cells stably are transfected with the human GABA_{C} ρ1 subunit for their electrical response to GABA and for their binding of GABA. FIG. 7 shows a representative GABA-elicited response recorded from one of these cells. The response is robust and exhibits the slow kinetics typical of GABA_{C}-mediated responses. GABA_{C}-expressing neuroblastoma cells and control, non-GABA_{C}-expressing neuroblastoma cells (ATCC) were analyzed for binding of (³H)GABA in a competition binding assay [incubation with fixed amount of (³H)GABA and varying amounts of non-radiolabeled GABA] using procedures similar to those described by Turek et al., 2002). Cells were seeded on 6-well plates and grown to 100% confluence, and then washed with 2 ml of binding buffer (50 mM Tris-HCl and 2.5 mM CaCl₂, pH 7.4) for 30 min. Fresh binding buffer (600 μl) containing 10 nM (³H)GABA in the presence of varying concentrations of unlabeled GABA (0-400 μM) was added, and the solution was incubated on ice [to minimize cellular uptake of the (³H)GABA] for 1 hr. After incubation, the plates were washed once with 2 ml ice-cold binding buffer, solubilized with 1 ml/well 0.3 N NaOH (shaking at room temperature for 10 min), and neutralized with 100 μl 3N HCl. The solubilized cells were then added to scintillation vials containing 10 ml Econo-Safe scintillation fluid and counted using a Beckman LS 6500M spectrometer. Nonspecific binding, defined as (³H)GABA binding observed in the presence of 400 μM unlabeled GABA, represented about 50% of the maximal level of total (³H)GABA binding observed in the absence of unlabeled GABA. FIG. 8 shows normalized levels (B/Bo, in percent) of specific (³H)GABA binding, i.e., normalized values obtained after the subtraction of nonspecific binding. The data yield a calculated IC₅₀ of 8.6×10⁻⁸ M for the non-radiolabeled GABA, and indicate workability of the (CH)GABA competition binding assay for determining binding properties of cell-expressed GABA_{C} receptors. Assay of the control cells indicated the absence of specific (³H)GABA binding (not shown).

Bacterial expression and ligand-binding of GABA_{C} extracellular domain in vitro reconstitution may employ, as a model target, solubilized GABA_{C} extracellular domain expressed using bacterial/baculovirus expression systems. The large extracellular N-terminal domains of GABA_{A} and GABA_{C} receptors are thought to contain the GABA-binding sites of the receptors. A primary objective is obtaining N-terminal extracellular domain of the human GABA_{C} ρ1 subunit. As shown in FIG. 10, alignment of the amino acid sequences of human ρ1 subunit, GABA_{A} receptor α1 subunit, acetylcholine binding protein (AchBP) and perch ρ1B predicts a GABA_{C} N-terminal core fragment (˜200 amino acids) structurally similar to AchBP and GABA_{A} [symbol] 1 (Cromer et al., 2002). To obtain a soluble form of this GABA_{C} core fragment, His-tagged fusion proteins of N-terminal sequences of human ρ1 and perch ρ1B GABA_{C} subunits (amino acid positions 68-273 for human ρ1; positions 64-269 for perch ρ1B) expressed these constructs in bacterial strain SG13009. These segments of ρ1 subunits were amplified by PCR and subcloned in-frame into the BamHI-HindIII site of the pQE-His vector (Qiagen), which contains the phage T5 promoter and a synthetic ribosomal binding site, RBSII, for high translation rates. Strain SG13009 contains the pREP4 plasmid code for the lac repressor protein that binds to the operator sequences on pQE vector and tightly regulates recombinant protein expression. When IPTG is added, it binds the lac repressor protein and allows the host cell's RNA polymerase to transcribe the sequence of the recombinant protein.

FIG. 10 depicts alignments of amino acid sequences for AchBP, GABA_{A} receptor al subunit, and human and perch GABA_{C} receptor subunits (GABA ρ1 subunits).

Proteins synthesized in bacteria were analyzed by electrophoresis under denaturing conditions (SDS/PAGE). FIG. 11 shows results obtained with expression of the human ρ1 construct in bacteria. No recombinant protein was observed in the uninduced cells (lane 1). With IPTG induction (0.2 mM for 3 hr at 37° C.), a prominent band of about 27 kDa was present in the sample prepared from whole bacteria (lane 2). Further analysis indicated that a majority of the synthesized recombinant protein was present in an insoluble form in inclusion bodies (lane 4) rather than as soluble protein in the supernatant (lane 3). Recombinant proteins were purified from inclusion bodies using the following protocol. After 3-hr induction with IPTG, cells were collected by centrifugation at 8,000 g for 10 min. Cell pellets were lysed by sonication (5 min, full power) in buffer (300 mM NaCl, 10 mM imidazole and 50 mM phosphate buffer, pH 8.0). Inclusion bodies (i.e., the pellet) were collected by centrifugation at 14,000 g for 1 hr. Inclusion body proteins were solubilized by sonication (5 min, full power) in buffer containing 6 M guanidinium HCl (GuaHCl), 500 mM NaCl and 20 mM NaPO₄, pH 7.4; the resulting suspension was subjected to ultracentrifugation (100,000 g, 1 hr), and the supernatant was filtered through a 0.22 μm membrane. The His-tagged recombinant proteins present in the supernatant were purified on a HiTrap HP chelating column charged with Ni²⁺ (Amersham Biosciences).

FIG. 11 shows SDS/PAGE analysis of recombinant His-human ρ1 protein synthesized in bacteria. Lane 1: uninduced cells. Lanes 2-4: induced cells; whole-cell lysate (2), supernatant (3) and pellet (4). Lane 5: protein standards.

To refold the His-ρ1B protein bound to the column, the following buffers were sequentially applied to the column: (1) 100 mM Tris (pH 7.5), 200 mM NaCl, IM L-arginine, and glutathione as a redox system (3 mM GSH+0.3 mM GSSG); (2) same as buffer (1) but without the redox components; (3) 100 mM Tris (pH 8.0), 500 mM NaCl and 0.5 M L-arginine; (4) 100 mM Tris (pH 8.0), 500 mM NaCl and 0.25 M L-arginine; (5) 100 mM Tris (pH 8.0), 500 mM NaCl and 0.1 M L-arginine; and (6) 100 mM Tris (pH 8.0), 500 mM NaCl. Elution from the column was performed using 100 mM Tris (pH 8.0), 500 mM NaCl, and 200 mM imidazole.

The eluted protein was subjected to dialysis against various buffers, as presented in the accompanying Table. Solubility was dependent on high pH (9.5-9.7), and the purified protein was finally dialyzed against buffers containing either Tris (50 mM), or CHES (15 mM) as buffering agents, pH 9.5, NaCl (20-200 mM) for subsequent analysis.

| Composition | | Protein | Composition | | Protein |
|---|---|---|---|---|---|
| of dialysis buffer | pH | Prep. | of dialysis buffer | pH | Prep. |
| 50 mM | 9.5 | Soluble | 50 mM | 9.5 | Soluble |
| NaPO₄, 500 mM | | | NaPO₄, 500 mM NaCl | 9.5 | Soluble |
| NaCl, 200 mM | 8.0 | Precipitate | 50 mM Tris- | 9.5 | Soluble |
| imidazole | | | HCl, 200 mM NaCl | 9.5 | Soluble |
| 50 mM | 7.8 | Precipitate | 50 mM Tris- | 9.5 | Soluble |
| NaPO₄, 500 mM | | | HCl, 20 mM NaCl | | |
| NaCl, 200 mM | | | 15 mM | | |
| imidazole | | | CHES, 200 mM NaCl | | |
| 50 mM | | | 15 mM | | |
| NaPO₄, 500 mM | | | CHES, 20 mM NaCl | | |
| NaCl, 0.5 mg/ml | | | | | |
| azolectin | | | | | |

Preliminary circular dichroism (CD) data obtained from the solubilized protein suggest an at least partially folded structure and argue against merely a randomly coiled state (FIG. 11; peak wavelength at λ=210-220 nm). This is consistent with the expected structure of the protein, based on comparison with the low-resolution structure of AcbBP (Brejc et al., 2001), which predicts a helical region and several β-sheet regions for the GABA_{C} extracellular domain. In addition, preliminary data obtained in two experiments (FIG. 13 and a second experiment) show that, by competition binding assay, the purified soluble protein exhibits specific GABA binding with an average calculated IC₅₀ of ˜3.5×10⁻⁸ M and average specific binding of about 70%. This is consistent with data for GABA-activation of human ρ1 receptors expressed in a neuroblastoma cell line, as determined in a competition binding assay with (³H)GABA (FIG. 8). These initial measurements of GABA binding by purified, soluble His-ρ1B protein suggest the feasibility of the in vitro reconstitution experiments proposed in Section D. A similar approach employing bacterial expression and isolation/solubilization of extracellular domain has been used successfully in studying both glutamate and glycine receptors (Chen & Gouaux, 1997; Breitinger et al., 2004). However, our data are preliminary, and variations seen in the radioligand binding results suggest that the bacterial protein expression/preparative procedures used here will require further optimization. The bacterial protein may improve the efficiency of protein re-folding by modifying the procedures according to published protocols (Chen & Gouaux, 1997; Breitinger et al., 2004; Oganesyan et al., 2004). In addition, experiments to determine biochemical/pharmacological properties of the soluble ρ1B protein are described in Section D. The FIG. 13 data, which suggest a GABA-binding affinity of order similar to that of the GABA dissociation constant determined for cell-expressed GABA_{C} implies the capacity of the extracellular domain for proper folding. As GABA-binding sites of native GABA_{C} receptors are thought to be located at junctions of the extracellular domains of adjacent subunits, as in acetylcholine receptors (Karlin, 2002; Cromer et al., 2002), significant GABA-binding activity may be an indirect indication of subunit oligomerization to form a homopentamer.

FIG. 12 depicts a CD spectrum of a preparation of soluble extracellular domain of perch His ρ1B in 10 mM NaCl and 15 mM CHES, pH 9.5. FIG. 13. (³H)GABA competition binding data obtained with a soluble His ρ1B preparation. Data points are averages of duplicate samples.

Screening of phage display peptides with GABA_{C}-expressing cells MAY employ phage display to identify 12-mer peptide sequences that can serve as an NNP anchoring element. GABA_{C}-transfected neuroblastoma cells have yielded sequences of peptides that preferentially bind to GABA_{C}-expressing cells. For phage selection, we used a screening method similar to that previously used to identify phages that bind to ErbB receptors (Stortelers et al., 2003). Briefly, 2×10¹⁰ phages (Ph.D-12 library from New England Biolabs, MA) were incubated with control, non-transfected neuroblastoma cells in binding buffer (PBS containing 0.2% BSA, 0.05% Tween 20) for 2 hr. Non-bound phages were collected and then incubated with GABA_{C}-transfected neuroblastoma cells for 2 hr. After rinsing several times with washing buffer (0.05% Tween 20 in PBS), bound phages were eluted using an acidic glycine buffer (50 mM glycine, 150 mM NaCl, pH 2.7) and neutralized with 1 M Tris, pH 8. After phage titration of the eluate, we performed a second and then a third round of bio-panning using the GABA_{C}-transfected neuroblastoma cells. After the third round of panning, DNA isolated from individual phage plaques was sequenced. The Table at the left shows the peptide sequences of two distinct groups derived from multiple phages. A highly conserved sequence was observed for each group. The 7 illustrated sequences represent individual phage clones from a total of 36 sequenced clones.

Phage ID
Group A

| | |
|---|---|
| 9 | H E T A V R Q T S P P M |
| 11 | H E T A C R Q T S P P M |
| 20 | H E T A V R Q T S P P M |
| 22 | H E T A V R Q T S P P M |

Group B

| | |
|---|---|
| 6 | H P K Q S L H F P D L S |
| 4 | H P Y D S L H F P R M S |
| 6-1 | H P Y D S L H F P R M S |

Visualization of receptor binding with nanocrystal-conjugated muscimol. A prototype system for testing candidate effectors may use prepared muscimol tethered via an aminocaproyl and PEG 3400 linker to AMP™ CdSe nanocrystals (coupling chemistry similar to that described by Rosenthal et al. (2002). The resulting muscimol-PEG-nanocrystal conjugate, which possesses an estimated 100-150 tethered muscimols per nanocrystal, is here abbreviated M-PEG-nc. By confocal microscopy we analyzed the interaction of M-PEG-nc with Xenopus oocytes expressing GABA_{C} receptors. Images were obtained from oocytes positioned in a glass-bottom dish and immersed in Ringer solution containing the test agent. Oocytes were bathed in a surrounding drop (25 μl) of 34 nM M-PEG-nc (i.e., 34 nM in nanocrystals) in Ringer solution for defined periods and then imaged or, as controls, similarly incubated with unconjugated nanocrystals. Other preparations were pre-incubated for 15 min with 34 nM unconjugated nanocrystals, with 34 nM of PEG-conjugated nanocrystals (lacking muscimol), or with 500 μM GABA prior to 5-min incubation with 34 nM M-PEG-nc. Fluorescence was visualized using a Leica DM-IRE2 confocal microscope (20× objective) with excitation at 476 nm. Fluorescence emission was detected over a wavelength interval (580-620 nm) that included the nanocrystal emission peak (λ=605 nm). Microscope settings relevant to detection of fluorescence emission were established at the beginning of experiments on a given day, and maintained without change for that set of measurements. The set of measurements (set 1 or set 2) performed on a given day employed a single batch of oocytes and a single preparation of M-PEG-nc. FIG. 14 (upper row) shows results obtained from oocytes expressing perch ρ1B GABA_{C} receptors (1) (set 1) and human ρ1 GABA_{C} receptors (2) (set 2), and from a non-injected oocyte (3) (set 2), upon 5-min incubation with medium containing 34 nM M-PEG-nc. For (1) and (2), the fluorescence image (left-hand side) shows a thin halo of fluorescence at the oocyte surface, the intensity of which exceeds the surround fluorescence. By contrast, only diffuse surround fluorescence was observed with the non-injected (i.e., GABA_{C}-lacking) oocyte (3). To illustrate the focus of the oocyte under investigation, panels 1-3 include (right-hand side) a bright-field image of the oocyte obtained simultaneously with the fluorescence image. The middle and lower rows of FIG. 14 [oocytes expressing, respectively, perch ρ1B receptors (set 1) and human ρ1 GABA_{C} receptors (set 2)] show results obtained on incubation with unconjugated nanocrystals alone (panel A); on pre-incubation with unconjugated nanocrystals followed by incubation with M-PEG-nc (B); on incubation with PEG-nanocrystals (lacking muscimol) alone (C); and on pre-incubation with GABA followed by incubation with M-PEG-nc (D). The data of A-B indicate the inability of unconjugated nanocrystals to bind to the oocyte membrane or to significantly inhibit M-PEG-nc binding; those of C indicate little if any binding by PEG-nanocrystals lacking muscimol; and those of D indicate that GABA blocks M-PEG-nc binding. M-PEG-nc binding was similarly blocked by pre-incubation with 500 μM muscimol (data not shown).

The upper row of FIG. 14 depicts oocytes expressing perch ρ1B GABA_{C} (1) or human ρ1 GABA_{C} (2), and non-injected oocytes (3) were incubated with 34 nM of muscimol-conjugated nanocrystals (M-PEG-nc) for 5 min. To the right of fluorescence images 1, 2 and 3 are corresponding brightfield images. Middle and lower rows: fluorescence images obtained with perch ρ1B (middle) and human ρ1 (lower) GABA_{C}-expressing oocytes. A: incubation with 34 nM of unconjugated nanocrystals for 15 min. B: oocytes pre-incubated with 34 nM of unconjugated nanocrystals for 15 min, removed from the pre-incubation dish, and then incubated with 34 nM M-PEG-nc for 5 min. C: oocytes incubated with 34 nM of PEG-nanocrystals (i.e., no conjugated muscimol) for 15 min. D: oocytes pre-incubated with 500 μM of GABA for 15 min, removed from the pre-incubation dish, and then incubated with 34 nM M-PEG-nc for 5 min.

Postsynaptic membrane receptors of the ligand-gated ion channel (LGIC) family mediate signal transmission at numerous types of chemical synapses in the central nervous system (CNS). In neural diseases that at a given synapse involve dysfunction/deterioration of the presynaptic neuron but preserve normal structure and function of the postsynaptic neuron, a possible approach to restoring signaling activity in the postsynaptic cell is to derivatize the postsynaptic receptor protein with a chemical structure that can regulate receptor activity in response to an external signal. Chemically modified LGICs with functional properties may restore or regulate neural signaling in neurodegenerative diseases. Receptors expressed in Xenopus oocytes and mammalian cell lines may be used as model systems. One such model system may be the GABA_{A} receptor, a heteromeric LGIC that is widely distributed in CNS tissue, is a target of drug therapy in CNS disorders. A key objective here may be the determination of specific sites on native GABA_{A} subunits that may accommodate the covalent attachment, by photoaffinity labeling, of chemical structures whose distal components exhibit controllable reactivity at the receptor's GABA- or benzodiazepine-binding sites.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 depicts signal transmission in a normally functioning synapse.

FIG. 2 illustrates a nanoscale neuromodulating platform.

FIG. 3 illustrates an iterative development process.

FIG. 4 depicts muscimol-biotin.

FIG. 5 illustrates muscimol-biotin high performance liquid chromatography.

FIG. 6 depicts agonist activity at GABAc receptors.

FIG. 7 illustrates agonist activity at GABAa receptors.

FIG. 8 graphs whole-cell patch recording of GABA elicited response.

FIG. 9 graphs (3H) GABA competition binding.

FIG. 10 illustrates the alignment of human amino acid sequences and perch P1B.

FIG. 11 illustrates expression of human ρ1 construct in bacteria.

FIG. 12 depicts circular dichroism from a protein.

FIG. 13 graphs specific GABA binding of a protein.

FIG. 14 illustrates results from oocytes expressing GABA_{C} receptors.

FIG. 15 depicts photoisomerization of azobenzene.

FIG. 16 illustrates photoregulated presentation of an agonist effector to the GABA receptor.

FIG. 17 depicts preparation of chain-derivatized muscimol.

FIG. 18 depicts a synthetic route to muscimol-azobenzene-PEG assemblies.

FIG. 19 depicts phosphinic acid analog of GABA.

FIG. 20 depicts the design of PEG-linked bivalent effectors.

FIG. 21 depicts a synthetic route to Y-shaped PEG-length effectors.

FIG. 22 depicts known photoregulated nAchR agonist.

FIG. 23 depicts solitary bipolar cells isolated from baboon retina.

FIG. 24 diagrams development approaches.

FIG. 25 depicts phage screening.

FIG. 26 depicts interactions of phage-derived peptide with GABA receptor.

FIG. 27 depicts the N-terminal region of AchBP with predicted solvent accessible surface areas.

FIG. 28 illustrates posterior probability analysis of amino acid substitution rates.

FIG. 29 depicts a scaffold approach.

FIG. 30 depicts synthetic routes to target push-pull azobenzene and derivatives through nitro-anilino coupling and diazonium coupling.

FIG. 31 depicts schematically operation of the NNP.

FIG. 32 depicts GABAa functionalization.

FIG. 33 depicts two LGIC receptor based therapies.

FIG. 34 depicts an alternate embodiment of the present invention.

### DETAILED DESCRIPTION OF THE PREFERRED EMBODIMENTS

The following description of the preferred embodiment(s) is merely exemplary in nature and is in no way intended to limit the invention, its application, or uses.

Expression/isolation of GABA_{C} extracellular domain and full-length sequences. The in vitro reconstitution of NNP components may employ isolated (i.e., purified) target GABA_{C} in the form of solubilized or membrane-associated full-length protein, and soluble extracellular domain. These in vitro experiments will complement electrophysiological and cell-based binding experiments, will provide information on the key issue of whether an activity of the test component determined in the whole-cell experiments reflects the test component's direct interaction with GABA_{C}. Isolated GABA_{C} may be obtained in the extracellular domain because monomers of Isolated GABA_{C}, like those of acetylcholine binding protein (AchBP) and of homologous extracellular domains of related membrane proteins, will spontaneously associate to form a pentameric complex whose extracellular topology and GABA-binding properties resemble those of homomeric GABA_{C} receptors in situ. Choice of sequence: The primary construct to be used to obtain GABA_{C} extracellular domain is a core extracellular segment of human GABA_{C} ρ1 subunit identified below. Because we have already succeeded in solubilizing the bacterially expressed perch ρ1B construct, we will employ the perch sequence as an alternative if difficulties are encountered with preparation/characterization of the human ρ1 protein. As perch and human GABA_{C} receptors exhibit similar pharmacology (Qian et al., 1998; Qian & Ripps, 2001), the expressed/solubilized perch sequence should be adequate for use in testing platform components. The N-terminal positions of both the human and perch constructs correspond with the beginning of a predicted GABA_{C} helical domain associated with a known helical domain of AchBP. In addition, these two expressed GABA_{C} sequences include a region inferred from mutation studies to contain the GABA-binding site for both GABA_{A} and GABA_{C} receptors (Chang & Weiss, 2000, 2002; Newell & Czajkowski, 2003; Sedelnikova et al., 2005). The C-terminal of both constructs corresponds with the C-terminal of AchBP and is the start of a putative transmembrane segment of native GABA_{C}.

Bacterial expression and protein refolding His-tagged fusion proteins have been generated with the extracellular domain of the human and perch ρ1 subunits. Both constructs are actively synthesized in bacteria in insoluble form, and can be purified in their denatured condition. For perch ρ1B protein, we have found a refolding condition that yields a soluble protein with potentially high GABA-binding capacity. To further characterize the purified protein, size exclusion and sucrose density centrifugation experiments may determine the molecular mass of the protein complex, which will serve as an index of oligomerization. The functional integrity of the purified protein will be determined by GABA-binding assays (see below). In addition, we will test modified procedures that could improve the refolding yields. Multiple parameters, including ionic strength, pH, the presence of redox agents, polar/nonpolar agents, poly(ethylene glycol) (PEG) and detergents are known to alter the refolding process (Chen & Gouaux, 1997; Breitinger et al., 2004). A systematic protocol to test the refolding efficiency of the agents may be used. The amount of soluble protein will be determined by SDS/PAGE, and functional integrity may be assayed by GABA binding.

Tests of GABA-binding activity and oligomerization state radiolabeled GABA may be used to determine the protein's GABA-binding activity in saturation binding assays [dependence of bound ³H on the molar concentration of (³H)GABA of fixed specific radioactivity] and in competition binding assays [dependence of bound ³H on the molar concentration of unlabeled GABA combined with a fixed amount of (³H)GABA]. Methods to be used to analyze (³H)GABA binding by the soluble protein may follow those described by Kim et al. (1992). Briefly, for saturation binding assays the protein will be incubated with varying concentrations of (³H)GABA at room temperature for 40 min, then vacuum-filtered through GF/B glass fiber filters (pre-treated with 0.5% polyethylenimine for 1 hr) to trap the protein. The filters may be rapidly washed once with 3 ml ice-cold binding buffer; bound protein will be solubilized with 0.3N NaOH and then neutralized with HCl; and bound (³H)GABA will be measured by liquid scintillation counting. Procedures for determining nonspecific (³H)GABA binding in these assays will be similar to those described in section C.3. Data interpretation: In competition binding assays, a GABA IC₅₀ for GABA_{C} extracellular domain similar to that of cell-expressed GABA_{C} may be interpreted as an indication of proper folding of the extracellular domain and used as the main performance criterion for this preparation. Furthermore, as the GABA-binding sites of native GABA_{C} receptors are thought to be located at the junctions of (the extracellular domains of) adjacent subunits, as in acetylcholine receptors (Karlin, 2002; Cromer et al., 2002), significant GABA-binding activity would be an indirect indication of subunit oligomerization to form a homopentamer. However, it is possible that even correctly folded and oligomerized extracellular domain exhibits GABA-binding affinity well below that of native receptor due to differences from native orientation/conformation of the associating subunits. GABA-binding activity will also be used to track appearance of the protein in chromatographic column fractions and to optimize protein preparative procedures (e.g., determining the effects of detergent treatment on protein recovery). Conventional methods of size-exclusion chromatography, native gel electrophoresis and dynamic light scattering will also be used specifically to determine whether the expressed extracellular domain forms a pentamer. Atomic force microscopy (AFM) may be used to investigate the expressed extracellular domain's state of oligomerization. Resolving monomer (predicted particle size: 40 Å) from pentamer (predicted outer diameter of the putative doughnut-shaped structure: 80 Å) is well within the capabilities of this method. AFM in tapping mode may be used to quantitatively analyze the sizes of GABA_{C} extracellular domain particles tethered to a supporting surface under defined conditions of GABA_{C} concentration (areal density of the protein), presence of added control protein of known size, and presence of surface-tethered organic compounds that modify the surface microenvironment, e.g., its hydrophilicity (e.g., Sharma et al., 2002, 2003). An important issue here will be the method used to tether the GABA_{C} extracellular domain to the supporting surface. It may prove workable to use a commercially available chip with epoxide activation or amine-reactive species (e.g., EDC technology similar to that used to cross-react proteins). However, these cross-linking approaches (or, e.g., terminal biotinylation of the protein and immobilization on an avidin-coated support) may yield heterogeneous orientation of the tethered protein (in the case of surface avidin coating, due to heterogeneous orientation of the avidin) that could confound determinations of the state of oligomerization. If these tethering approaches prove to be problematic, GABA_{C} may be tethered using a more site-selective procedure (C-terminal histidine-tagging of the protein and tethering to a Ni²⁺ support, or cysteine-tagging and tethering to a gold surface) to achieve greater uniformity in protein orientation. In summary, GABA-binding activity similar to that of the native receptor, and the occurrence of pentameric structure as determined by chromatographic behavior and AFM, together with CD and SDS-PAGE behavior, will together represent performance criteria for the extracellular domain preparation.

Expression of full length and extracellular domain GABA_{C} in baculovirus system. Baculovirus (i.e., insect cell) expression of full-length GABA_{C} may yield enriched protein that is folded and associates to form a pentameric structure. Relative to bacterial expression, a greater likelihood of correct folding is expected in the insect cell line (due to the presence of ER, chaperone proteins and folding machinery) even if the protein being expressed is extracellular domain rather than full-length. Preparative procedures to be used are based on experience with use of the baculovirus system for membrane protein expression [e.g., Stauffer et al., 1991; Gatto et al., 2001]. In particular, the expression of mammalian membrane proteins has been successfully achieved by the infection of sf9 or High Five cells with recombinant baculovirus particles; membrane proteins that have recently expressed and whose molecular characterization continues includes Na, K-ATPase, a heterodimeric active transport protein, Wilson Disease protein (i.e., ATP7B, a human Cu-activated transporter), and hCTR1 (the major human membrane protein responsible for Cu entry into cells; Hu & Kaplan, 2000; Eisses & Kaplan, 2002; Tsivkovskii et al., 2000; Laughery et al., 2003). In the case of the Na,K-ATPase, a protein not normally present in sf9 cells, baculovirus-mediated expression produces the protein at levels representing 3-5% of total membrane protein, a level significantly higher than obtainable in mammalian cells. Moreover, the expressed protein exhibits catalytic activity similar to that of the protein expressed in mammalian cells, i.e., this two-subunit protein properly assembles and exhibits full functionality when expressed in the insect cells. Strategies that have proven successful for other membrane proteins to express GABA_{C} receptor in sf9 membranes may be used. Overexpression will supply a source of intact full-length receptor, and functionality of the receptor will be confirmed by electrophysiological (patch-clamp) recording. As well as using recombinant baculovirus, we also will prepare sf9 insect cells to stably express the GABA_{C} receptor. The approach of preparing stably expressing sf9 cells is one that we have used successfully for CTR1. We also will use the baculovirus system to produce GABA_{C} extracellular domain in the event that bacterial expression of the protein does not yield re-folded functional protein in quantities sufficient for the proposed studies. This approach will involve the engineering, preparation and isolation of recombinant baculovirus; the infection of insect cells and their fractionation; and techniques associated with isolation of the expressed receptor molecules. The most critical of these steps are the construction of recombinant virus particles, the maintenance/infection of sf9 and High Five cells, cell membrane fiactionation, Western blot analysis, and immunoprecipitation procedures. Briefly, donor plasmids will be constructed by subcloning wild-type GABA_{C} receptor into one of the cloning sites of the pFASTBACDUAL vector. Recombinant baculovirus may then be produced following the Bac-to-Bac baculovirus expression system provided by the manufacturer (Life Technologies, Inc). The best MOI values and periods of infection prior to cell harvesting will be determined for GABA receptor expression. The full-length receptor will appear in membrane fractions and its distribution among the plasma membrane, ER and Golgi pools will be determined through assays of GABA-binding. This will enable us to determine (in ligand-binding experiments) whether there are functional differences in the receptor in each fraction. If no such difference is detected, unfractionated membrane preparations may be used. Mutant GABA_{C} receptors (for example, with site-directed modification) can also be generated using these protocols. We anticipate that for isolation of the extracellular domain, we will express an epitope-tagged version bearing the His6-epitope at the C-terminus, to facilitate purification with metal-ion columns as was done with the recent successful expression/isolation/purification of the ATP-binding domain of the Na,K-ATPase in the Kaplan laboratory (Gatto et al., 1998; Costa et al., 2003). For use in the reconstitution assays, we will investigate the preparation of both membrane-associated and solubilized (by e.g., CHAPS) full-length protein (e.g., Stauffer et al., 1991) and adopt the more readily obtained preparation for routine use. In the event of difficulties with expression of the extracellular domain sequence in the baculovirus system, an available alternative strategy is to express, in this system, a mutated full-length sequence containing an engineered protease site. The needed size of the introduced cleavage site is likely to be about 10-15 amino acids (including, e.g., glycines and prolines as well as the specific amino acids needed for recognition by the protease) to displace the desired extracellular domain from the surface of the plasma membrane, i.e., to make it accessible to the protease. In addition, for protein purification, we can engineer the cleavage site to incorporate adjacent histidines (for attachment of the protein to a nickel-coated substrate) or cysteines (for attachment to a gold substrate) (e.g., Gatto et al., 1998). More generally, a further alternative strategy for obtaining purified membranes containing full-length GABA_{C} is to use an already available neuroblastoma cell line stably transfected with GABA_{C} human ρ1 subunit.

Crystallization: Obtaining structural information on the GABA_{C} extracellular domain would greatly benefit interacting molecular structures with this domain. In light of the importance of such information (e.g., Sabini et al. 2003), we crystallize the putative pentameric complex of GABA_{C} extracellular domain prepared from bacterial and/or baculovirus expression systems. Crystallization methods needed for this pilot study are well established. As GABA_{C} ρ1 subunits are predicted to form a homopentamer, purified GABA_{C} extracellular domain should afford crystallization of pentameric complexes. To increase the likelihood of obtaining diffraction-quality crystals, we will test GABA_{C} fragments of different lengths and from different species. Crystallization procedures will employ pre-formulated solutions (Hampton Research) and use of differing protein concentrations and temperatures (4, 12 and 20° C.). An available rotating-anode x-ray generator and image plate detector, may be used to screen any crystals that attain a suitable size (˜100-200 μm). This procedure solves the structure by molecular replacement using the available model of AchBP (Brejc et al., 2001; Cromer et al., 2002). If AchBP proves to be an insufficiently correct model, the structure may be solvable de novo using the Multiwavelength Anomalous Dispersion technique.

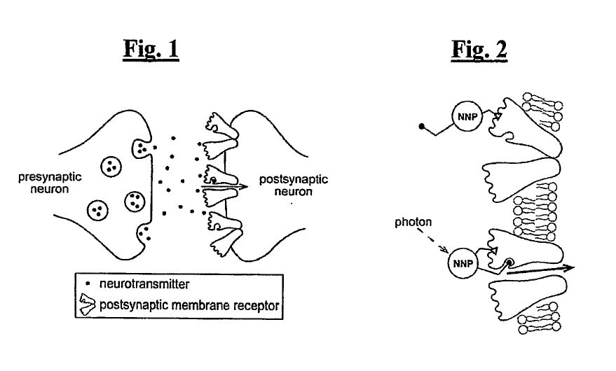

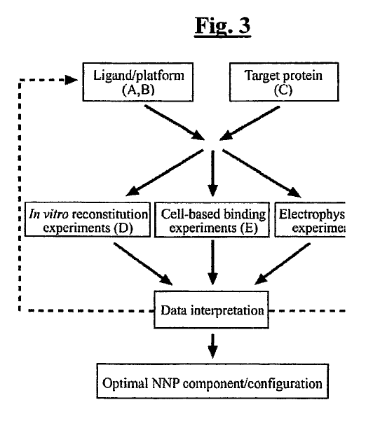

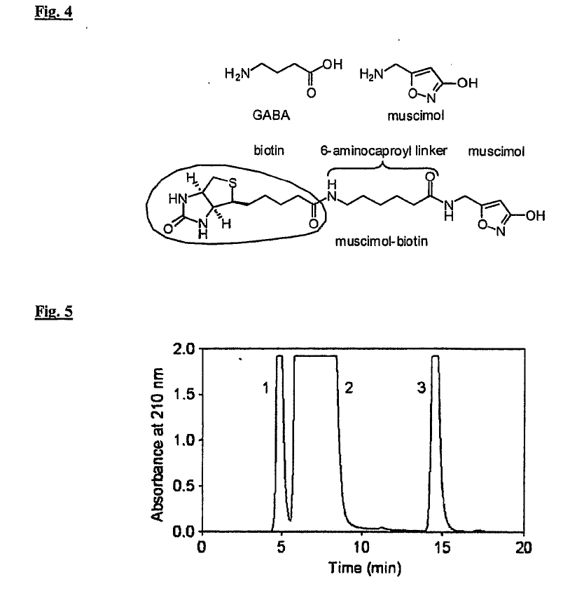

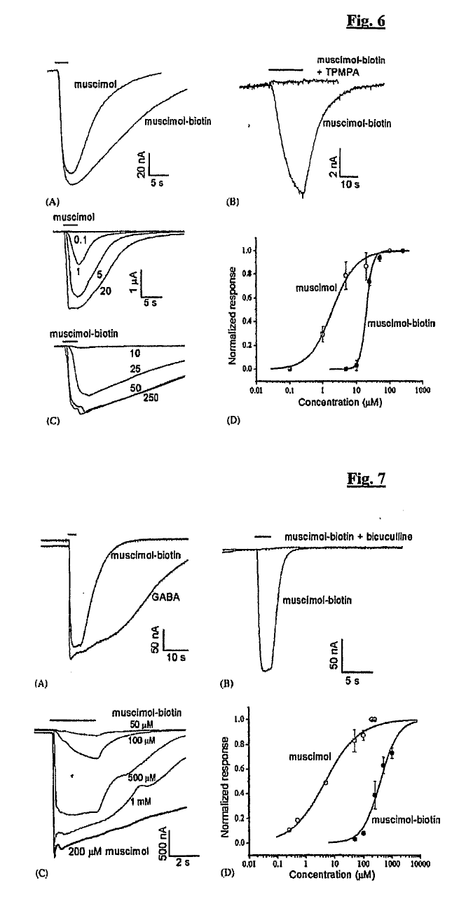

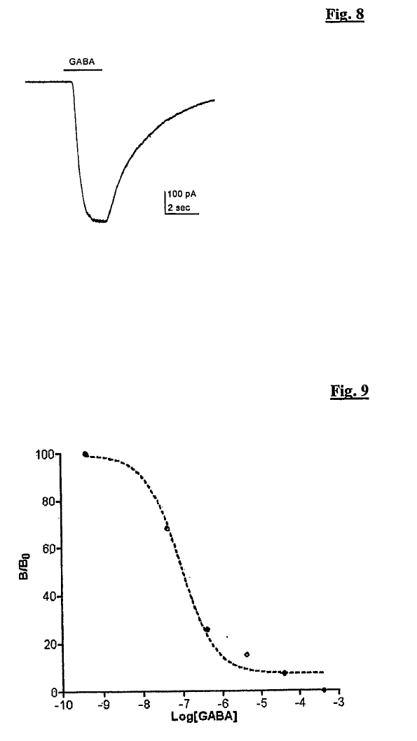

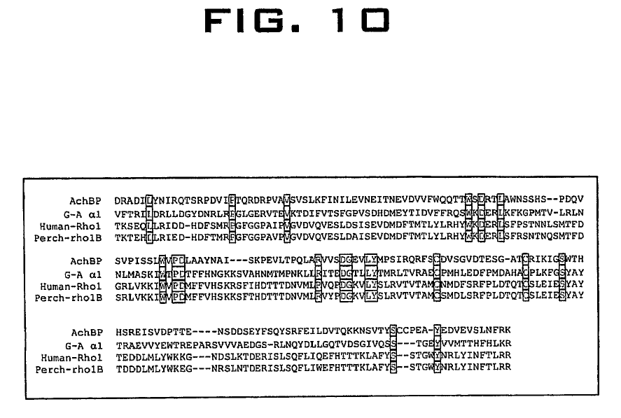

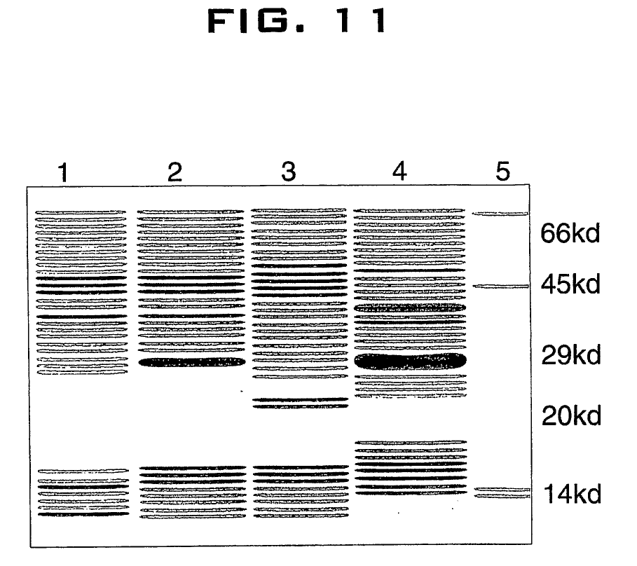

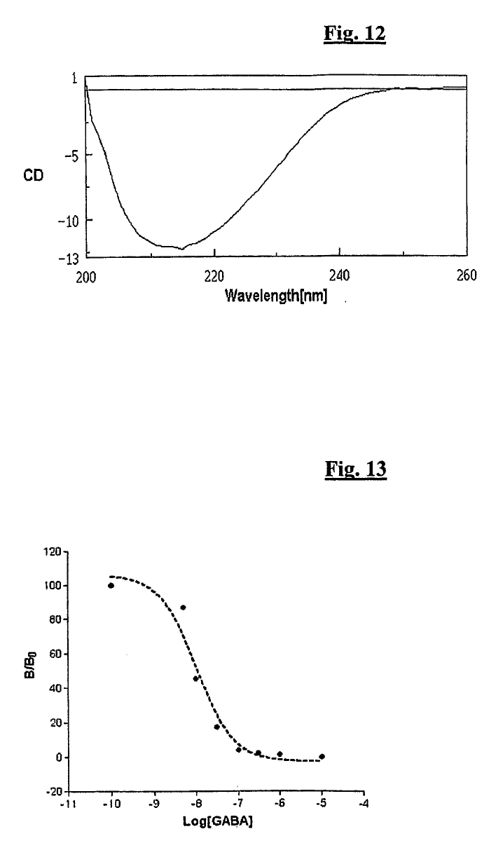

GABA_{C} effectors. Tetherable, i.e., chain-derivatized, compounds that have activity at the GABA_{C} receptor, will, upon coupling with photoswitch/anchor components, afford light-regulated control of receptor activation (cf. FIG. 1). There may be two strategies, both of which involve positioning a photoisomerizable organic structure in close proximity to the effector moiety. The following sections address, sequentially: the rationale for using azobenzene as a prototype photoswitch; the syntheses of candidate compounds that incorporate an effector, neighboring photoswitch, and poly(ethylene glycol) (PEG) linkers; and approaches for biophysical/electrophysiological testing of the synthesized structures.

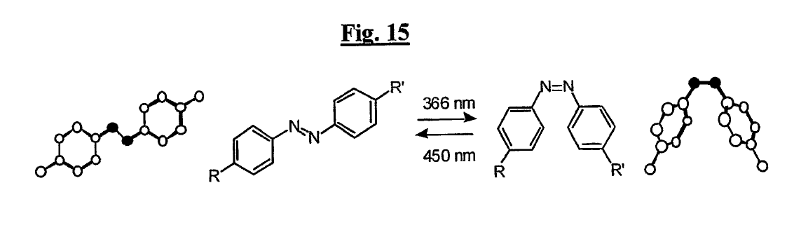

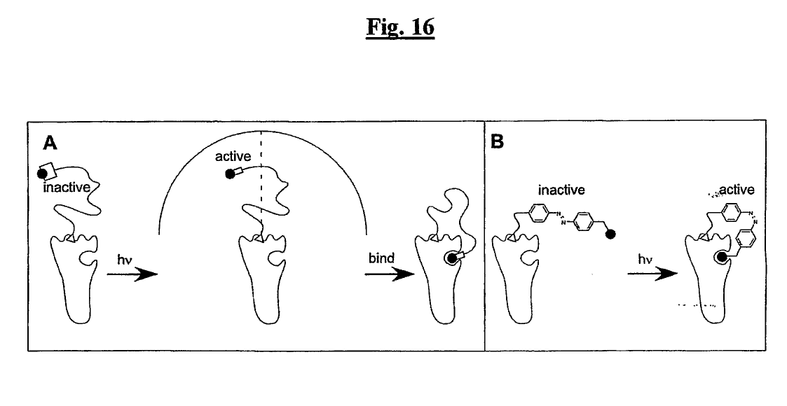

Rationale for use of azobenzene-based photoswitches: Azobenzenes, which have been used widely as photochemical switches, undergo cis/trans isomerization of the N═N bond in response to light. At thermodynamic equilibrium in darkness, azobenzenes exist almost exclusively in the trans form. Isomerization to the cis form is induced by near-UV light (366 nm), and back-isomerization to trans is induced by visible light. The photoisomerization event is rapid (˜1 ps), and population changes are readily accomplished on a sub-millisecond time scale with a flashgun or laser apparatus (Lester & Nerbonne, 1982; Gurney & Lester, 1987; also cf. Denk, 1997). The trans and cis isomers of azobenzene differ in two important respects. The first is geometric: the trans configuration is planar and provides a large, flat hydrophobic surface, whereas the cis configuration is forced out of planarity by steric clashes between the rings, giving it a bulky, irregular shape (FIG. 15). The second difference is electrostatic: the trans configuration has no net dipole moment due to the cancellation of internal dipoles through symmetry, while the cis configuration has a large dipole moment that makes it more polar and less hydrophobic. FIG. 15 depicts photoisomerization of azobenzene. The trans to cis isomerization decreases the distance between the 4- and 4′-substituents (R and R′) from 12 Å to 6 Å.

Azobenzenes have several additional advantages. Chief among these are small size, predictable geometry, ease of synthesis, chemical robustness, tolerance for a wide array of substituents, and relative absence of photochemical side reactions. Moreover, Lester et al. (1980) have linked an azobenzene-based analog of acetylcholine directly to the acetylcholine receptor and demonstrated light-regulated receptor activation, and Banghart et al. (2004) have very recently employed azobenzene as a switch to photo-regulate the activity of a mutant K⁺ channel. In the parent azobenzene itself, and in most simple derivatives, the cis isomer is produced by irradiation in the near-UV (370 nm), and back-isomerization to trans is effected by blue light (450 nm), and the dark isomerization is extremely slow (days). Importantly, the isomerization wavelengths can be red-shifted such that both are in the visible range, and the thermal isomerization greatly accelerated through the use of special substituents, notably electron donor groups on one ring coupled with electron acceptor groups on the other, so-called “push-pull” azobenzenes. The slow thermal isomerization of typical (not push-pull) azobenzenes is a great advantage in characterizing the behavior of the individual photoisomers, whereas the rapid thermal isomerization will be necessary in a working device.

System design and performance criteria: Synthesized chain-derivatized effector compounds found in free (i.e., untethered) form to have activity at GABA_{C} receptors will become candidates for anchoring and photoswitch incorporation, for further testing as workable NNPs. Identification of an effector as a candidate for use in the ultimately desired NNP will be based on the GABA_{C}-binding properties of the effector (free effector, or part of an effector/photoswitch/linker assembly): specifically, the dissociation constant (K_{D}) determined in cell-based and in vitro binding assays; the EC₅₀ (or IC₅₀) determined by measurement of the dose-response curve in electrophysiological experiments; and, for effector/photoswitch/linker assemblies, length of the linker chain and photoisomerization-induced change in end-to-end photoswitch length. FIG. 15 illustrates two models through which the suitability of an agonist effector will be estimated from the interrelationship of these four parameters. Here, for simplicity, we consider the case of an agonist effector (e.g., muscimol), a linker consisting of a linear PEG chain, and azobenzene as the photoswitch.

A: Strategy 1 (long linker): The effector (filled circle), close-coupled to an azobenzene photoswitch (open rectangle), is anchored (open triangle) to the receptor via a long, highly flexible PEG linker. The “inactive” isomer of the photoswitch (denoted by the large size of open rectangle) conforiationally blocks effector binding. Light, by isomerizing the photoswitch (transition to small open rectangle), relieves the conformational block and allows effector binding at the receptor's ligand-binding site. At all times the close-coupled effector-photoswitch is confined to an approximate hemisphere by the PEG linker, which has a random conformation. The size of the hemisphere is controlled through the length of the PEG chain, which is chosen to establish a local molarity of the effector-photoswitch greater than the EC₅₀ for the active state (active isomer of the photoswitch) and below the EC₅₀ for the inactive (i.e., non-binding or weakly binding) state. B: Strategy 2 (short linker): A constitutively active effector is prevented from reaching the receptor's ligand-binding site by the conformational constraint of the azobenzene photoswitch, which is anchored to the receptor by a minimal length of tethering chain (e.g., a few ethylene oxide units). Photoisomerization of the switch re-orients the effector, allowing its binding to the receptor's ligand-binding site. Molecular structures are not drawn to scale.

Local concentration of effector: Tethering the effector to the receptor causes an increase in the local concentration (molarity) of the effector, a point of key importance to NNP design. For the present discussion we consider PEG chains of different lengths. PEG is a highly flexible polymer, and a fully extended PEG chain has a length of 3.5 Å per EG unit. However, Bedrov & Smith (2003) showed that this fully extended configuration is energetically disfavored, and that the interval representing 0-80% of full extension is essentially isoenergetic. Thus, we will assume that the free terminus of a PEG chain, when the other end is attached to a membrane receptor, moves randomly about an isoenergetic, hemispheric volume with a radius equal to (n)(0.8)(3.5 Å), where n is the number of EG units (FIG. 16). PEG 3400 (n=77) provides an attractive starting length because a wide variety of functional derivatives of it are commercially available and moderately priced, and the size of the hemisphere (radius=216 Å) is larger than the GABA_{C} receptor subunit, so that a molecule tethered at any point on the receptor should have free access to the ligand binding site. In the simplest possible model, wherein the anchored effector is viewed as a freely diffusing element, the effective volume available to the effector is 2.1×10⁻²⁰ L, and its effective molarity is 79 μM. This simplest scenario ignores several potentially complicating factors, including: the volume excluded by the chain itself; a geometric factor influencing the effector's local concentration [i.e., proportionality to (radius)⁻² in non-excluded volume elements]; the non-planarity of the receptor's extracellular surface and surrounding membrane; possible attractive/repulsive interactions of the effector, photoswitch or PEG chain with the receptor or surrounding membrane; and the need for (and possible interactions among)>2 tethered effectors per pentameric receptor to achieve activation (Amin & Weiss, 1996; Karlin, 2002). The aggregate effect of these factors will need to be resolved through variation of the PEG chain length. This specifically predicts that an effector with an EC₅₀ substantially above about 80 μM will never achieve significant occupancy, whereas an effector with an EC₅₀ significantly below 80 μM will always have significant occupancy.

Strategy 1: Long PEG chain. Successful operation of the device requires a high differential in the binding affinity of the effector upon isomerization of the photoswitch. It is first helpful to consider the effect of the photoswitch on the effective volume calculation. A p,p′-disubstituted azobenzene moiety is approximately 12 Å long in the trans form and 6 Å long in the cis form (FIG. 15), and other conceivable photoswitches undergo changes of the same order of magnitude. Clearly, a 6 Å change in radius is negligible in relation to the 216 Å effective length of a PEG 3400 chain. Workability in the long-chain strategy requires that the photoswitch moiety be proximally coupled to the effector, so that it acts through local, specific effects such as steric hindrance (FIG. 16A). Specific performance criteria are dictated by the effective molarity of 80 μM enforced by the PEG 3400 chain. For the device to function well, the EC₅₀ of the permissive (active) photoisomer must be substantially lower than 80 μM, and that of the non-permissive (inactive) photoisomer must be significantly higher. These criteria define a target range of affinity for the permissive and non-permissive forms of the effector/photoswitch combination. That is, the permissive form should have an EC₅₀<25 μM, the non-permissive form should have an EC₅₀≧250 μM, and the dynamic range of the effector-photoswitch combination needs to be at least one order of magnitude. It is important to note that it is entirely reasonable to expect such a dynamic range from an azobenzene-based system. For example, Westmark et al. (1993) prepared a simple, azobenzene-based inhibitor of the protease papain which displayed Kᵢ's of ˜2 μM and ˜80 μM for the trans and cis forms, respectively (dynamic range of 40). In addition, the target affinity of EC₅₀<25 μM in the permissive form is also reasonable in that the amount of material required is not excessive, and a saturating response can be achieved at 100 μM (untethered ligand), which is below the point where water-solubility of the ligand is expected to be a problem. Importantly, we have already shown that muscimol-biotin has an adequate EC₅₀ (20 μM at GABA_{C}).

Strategy 2: Short PEG chain. This strategy relies on the use of expansion, contraction or bending of the photoswitch, coupled to both receptor and ligand with tethers of minimal length, to re-orient the effector moiety. FIG. 16B depicts the case in which the dark-state trans isomer precludes full entry of the effector into the ligand binding site. Photoisomerization to the cis form relieves the block and allows activation. It is useful to consider this strategy in relation to Strategy 1 discussed above, as the design parameters in Strategy 2 are completely different. First, the net length of the PEG chains employed is required to be short (n<6). In this regime, the effective molarity of the effector is in principle very high (over 10 mM), but its movement will be highly constrained by the short tether, and displacements of a few A within the photoswitch moiety are relied upon to move the effector into or out of the binding site. A specific (though hypothetical) implementation of the FIG. 15B scheme might involve an anchoring at 10 Å from the opening to the binding site, an azobenzene photoswitch, and a linker of two EG units. The maximum extension of this linker is 7 Å, and the minimum is about 3 Å (van der Waals contact of termini). As depicted in FIG. 15B, the range of extension of the short linker (3-7 Å), in combination with the rigid 12 Å trans azobenzene, precludes access of the effector to the binding site. By contrast, photoisomerization to a 6 Å cis azobenzene permits access.

Performance criteria for binding affinity: In principle, a very weak effector, i.e., one with a high value of EC₅₀, could be employed in Strategy 2 due to the high effective molarity envisioned. However, for Strategy 2 we nevertheless seek an EC₅₀ for the untethered effector of 100 μM or lower. One reason is that the effector could ultimately be responsible for targeting the NNP to the GABA_{C} receptor, and molecules of lower affinity might lack adequate specificity. Another reason is practicality, in that compounds with significantly higher EC₅₀'s must be made in greater quantities for characterization and might present solubility problems. Photoisomerization directionality: Both strategies 1 and 2 are intended to operate with trans-to-cis photoisomerization as the activating event, i.e., the cis form is permissive. Although a device functioning in the opposite way (trans form permissive) in vitro, is within the scope of the invention the trans-to-cis activation is preferable. Our reasoning is as follows. The thermodynamic preference for the trans form is large, ΔG≈ΔH=49 kJ/mol in azobenzene itself (Dias et al., 1992), leading to negligible thermal population of the cis state (molar ratio cis/trans=K_{eq}=3×10⁻⁹ at 25° C., derived from the relation K_{eq}=exp(−ΔG°/RT). Thus, a device with a non-permissive trans form will return spontaneously to the baseline dark state, whereas a device in which the trans form is permissive will spontaneously move toward full activation through thermal cis-to-trans isomerization. With the cis form permissive, binding affinity by the ligand will favor the cis configuration. However, for this effector-binding energy to overcome the intrinsic thermodynamic preference for trans, the cis form must have a binding energy of >49 kJ/mol, and hence a K_{D}<3 nM. As known GABA_{C} effectors have K_{D}'S well above this value, there should be no constraint on prototype system design by an upper-limit binding affinity in a trans-non-permissive configuration.

Candidate Effectors: The NNP employs an agonist as effector. Use of an antagonist effector would be difficult in vivo, as a background of GABA would be required. However, the identification of tetherable GABA_{C} antagonists could provide important insights into ultimate NNP designs and, in particular, could be valuable for development of a “scaffold” strategy for platform anchoring. Both agonists and antagonists as potential effectors are within one scope of the invention. For the agonist, we will rely on muscimol, as we have successfully prepared a tetherable derivative of it through simple modification chemistry, and this derivative has sufficient potency (Vu et al., 2005). To our knowledge, this success has not been paralleled (by ourselves or others) for other classes of GABA agonists, and the conceivable approaches to the synthesis of such compounds are considerably more labor-intensive than derivatizing muscimol. Thus, we will discuss only muscimol derivatives with the understanding that similar strategies will be applied to other classes of agonist as tetherable derivatives emerge. To prepare a tetherable antagonist, we will explore phosphinic acids, which are the only known specific GABA_{C} antagonists.

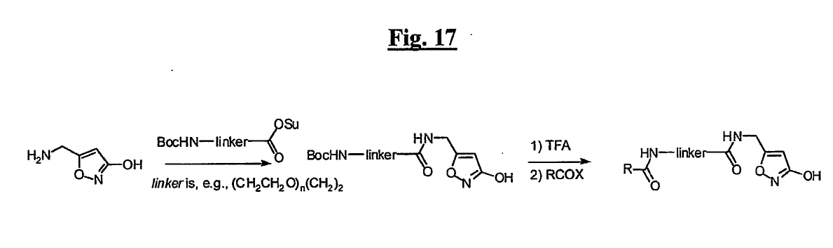

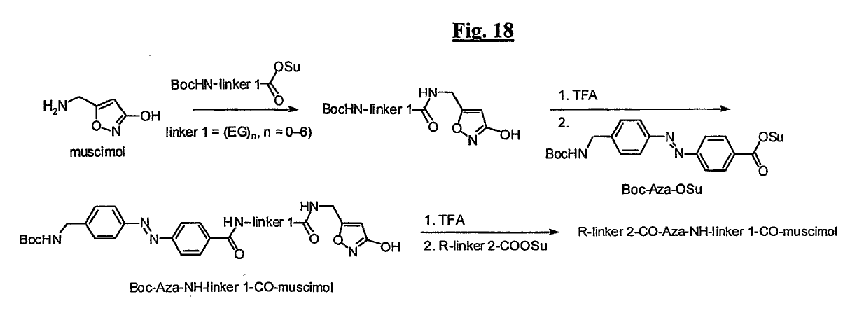

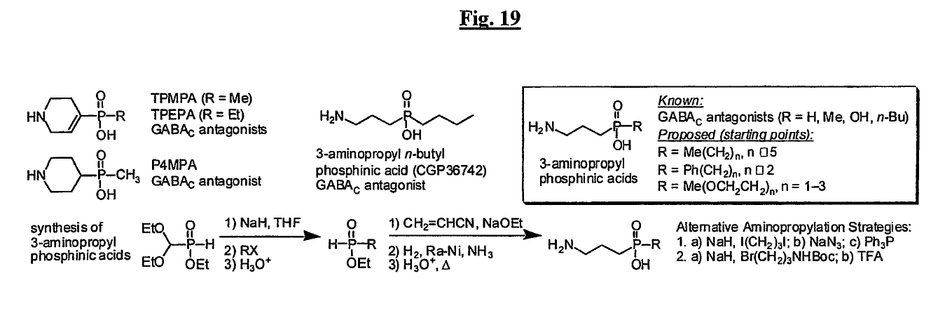

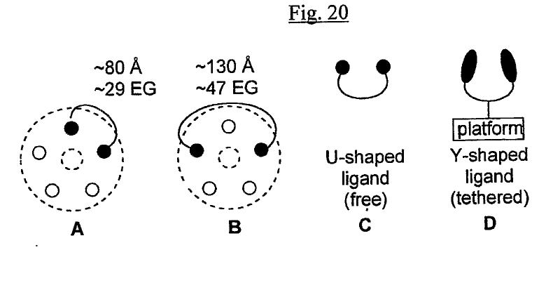

Agonist (muscimol) approach: The rationale for investigating muscimol derivatives is based on results obtained with muscimol-biotin and muscimol-BODIPY, two chain-derivatized forms of muscimol that exhibit agonist activity at both GABA_{C} and GABA_{A} receptors expressed in Xenopus oocytes [Vu et al., 2005 (Appendix 2); muscimol-biotin data summarized in section C.2]. The activities of these compounds show that muscimol conjugated to structurally different molecules through a linear (aminocaproyl) linker can activate these receptors. As pointed out in the Discussion section of Vu et al. (2005), it is not yet clear to what extent the biotin in muscimol-biotin, with its relatively short aminocaproyl linker, extends to the extracellular space beyond the receptor's ligand-binding site. However, preliminary fluorescence data indicate that muscimol conjugated to a (sterically bulky) CdSe nanocrystal via a PEG 3400-aminocaproyl combination linker displays marked affinity for GABA_{C}. For accessibility of the distal end of the chain, we prepare a series of compounds biotin-(PEG)ₙ-muscimol and assess the impact of soluble streptavidin on the biochemical and physiological properties of the compound. Where co-incubation with streptavidin lacks an effect, it is inferred that the distal end of the chain is both beyond the immediate vicinity of the binding site and is accessible to the bulky streptavidin protein; such linkers are ideal. Where streptavidin has an effect, it is inferred that the distal terminus is not free of the receptor; here, the corresponding chain lengths will potentially be useful positioning of the photoswitch. In addition to PEG 3400 (n=77 EG units), initially we will test n=4, 8, 16 and 32, relying, where possible, on commercially available bifunctional PEG derivatives and preparing unavailable reagents from appropriate base polymers (FIG. 17). These compounds will be purified by reversed-phase HPLC or (for PEG derivatives) crystallization or size-exclusion/ion-exchange chromatography. For muscimol-based effector/photoswitch/linker assemblies identified in tests of GABA-binding and electrophysiological activity, we will prepare variants that contain acyl-linked GABA rather than muscimol. Although a previously synthesized, biotinylated GABA-based compound exhibited little or no electrophysiological activity, these GABA-based variants may be useful in combination with anti-GABA antibody (Saifuddin et al., 2003) as a check on AFM surface characterization procedures to be used to study the interaction of GABA_{C} extracellular domain with muscimol-containing test components. In the event of problems with the preparation/activities of muscimol derivatives, an alternative may be trans-aminocrotonic acid (TACA) as an effector moiety (Kusama et al., 1993).

Effector-conjugated CdSe nanocrystals: CdSe nanocrystals (diameter 4-10 nm), either as uncoated cores or coated with a shell that passivates the core material and can itself be functionalized using conventional bioconjugate chemistry, exhibit the ability to present ligands to membrane surface receptors under physiological conditions (Rosenthal et al., 2002), and have several properties of particular value. The first of these is the ability to support a large and adjustable number of tethered ligands; that is, the maximum number of tethered test ligands (˜160 for a 60 Å CdSe nanocrystal) can be reduced by diluting the test ligand with a suitably functionalized inert ligand during conjugation. In addition, CdSe nanocrystals have high fluorescence yield (product of quantum yield and extinction coefficient) with excitation near 480 nm, and resistance to photobleaching. These properties, together with preliminary data indicating the feasibility of targeting cell-expressed GABA_{C} with PEG-linked, muscimol-conjugated nanocrystals, encourage their use as a prototype system for addressing two issues of importance to the proposed research. First, these nanocrystal preparations will afford an alternative test of “receptor clearance” by the linker component of a given derivatized ligand. That is, despite the presence of many copies of a given effector/linker conjugate on the nanocrystal, a linker whose ligand-distal (i.e., nanocrystal-linked) terminus is too short to extend beyond the receptor's extracellular surface is expected not to bind to the receptor. Second, these preparations afford the ability to examine the effect of a wide range of valencies of a test effector. Due to the multivalency of the GABA_{C} receptor, we anticipate that effector valency will be an important parameter to investigate. The synthesis of chemically defined divalent effectors may be used. Nanocrystal-conjugated effector preparations will allow a survey of the effect of valency through appropriate dilution of the effector by the co-conjugation of inert ligand to the nanocrystal. In this way, a wide range of average valencies can be prepared rapidly. While any given preparation will be heterogeneous (i.e., will contain a distribution of valencies with known average), correlation of the average valency with data obtained in fluorescence visualization and other in vitro and electrophysiological experiments will guide the choice of synthetic structures of defined valency.

Incorporation of a photoswitch into effector-PEG preparation involves positioning the photoswitch in close proximity to the effector (FIG. 16). In pursuit of these strategies, we construct a series of molecules in which muscimol is connected through a short linker (0-6 EG units, linker 1 in FIG. 18) to a photoswitch element (e.g., azobenzene-based amino acid), an optional second linker (linker 2) of varying length, and a variable distal group. FIG. 18 depicts a synthetic route to muscimol-azobenzene-PEG assemblies. Linker 1 is 0-6 EG units, linker 2 is 0-77 EG units, and Aza (Ulysse & Chmielewski, 1994) is a representative azobenzene-based amino acid. A first objective is to identify a functional photoswitch/linker combination. Initially we will examine linkers of 0-6 EG units and a series of azobenzene-based amino acids. In nine of these, including several based on azobiphenyl, the change in end-to-end distance produced by trans-to-cis photoisomerization (18 to 5 Å) is amplified relative to the corresponding change in azobenzene (12 to 6 Å) [Park & Standaert, 2001]. For prototype investigation of other, distally attached NNP components, we will next prepare a group of biotin-terminated PEG linkers. Electrophysiological and/or other testing of these molecules in the presence vs. absence of streptavidin will allow us to determine the lower limit on the length of the linker 2 chain that allows biotin to move clear of the ligand binding site. The occurrence, in the streptavidin experiments, of a differential result between cis and trans azobenzene isomers would be interpreted to suggest that the linker length under investigation is at the threshold of streptavidin accessibility to the biotin moiety, a finding that would facilitate determination of the clearance length of the linker/photoswitch assembly.

Alternative strategies: The following points describe alternative approaches to be pursued to identify GABA_{C}-reactive effector/photoswitch/linker assemblies. (1) Antagonist (phosphinic acid) approach: An alternative strategy within the scope of one invention is the use of phosphinic acids, a known prominent class of GABA_{C} antagonists. Phosphinic acid analogs of GABA; upper left: reduced-pyridine derivatives (TPMPA and TPEPA). Middle: 3-aminopropyl n-butyl phosphinic acid. Right: proposed new 3-aminopropyl phosphinic acids. Lower: General synthetic route to 3-aminopropyl phosphinic acids (Froestl et al., 1995). We have chosen 3-aminopropyl n-butyl phosphinic acid (CGP36742) (Chebib et al., 1997b) because it has an IC₅₀ of 60 μM (suggesting that K_{D}≦60 μM), which is below our 80 μM criterion, and because it demonstrates that a long alkyl side chain is tolerated in this series. By analogy with the derivatization of muscimol described above, the objective of the syntheses of phosphinic acid analogs is to identify PEG-chain-derivatized (i.e., tetherable) compounds that incorporate an antagonist effector and neighboring photoswitch, and that in binding and electrophysiological tests exhibit GABA_{C} reactivity. We begin by synthesizing, via Froestl's method (Froestl et al., 1995), a series of arylalkyl groups (starting with 2-phenylethyl) coupled with the 3-aminopropylphosphinic moiety to determine how long a chain is tolerated, and whether a sterically bulky group (initially, phenyl; subsequently, photoswitch candidates) is tolerated at the end of the chain, where the chain of interest is derived from the corresponding bromide or tosylate. Most of the requisite alkyl and arylalkyl bromides are commercially available, while the PEG tosylates are straightforward to prepare. Upon identification of a GABA_{C}-reactive phenyl derivative, we synthesize compounds with additional substituents on the benzene (e.g., alkyl group or amide) as potential tethering moieties.

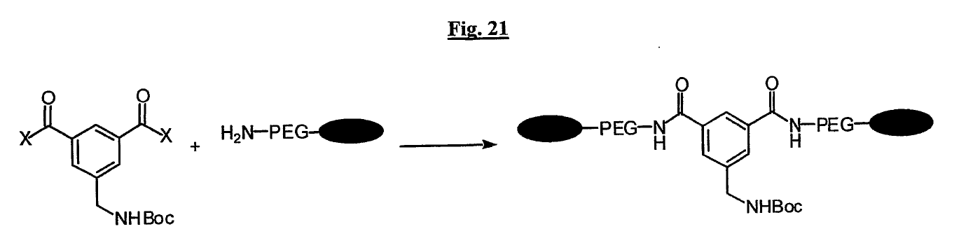

(2) Multivalent ligands: Native GABA receptors and other ligand-gated ion channels exist as heteromeric pentamers with two ligand-binding sites, and the full channel opening requires the simultaneous binding of two ligands (Woodward et al., 1993; Ortells & Lunt, 1995; Karlin, 2002). Moreover, homomeric GABA_{C} receptors are believed to exist as pentamers with five GABA-binding sites (one at the interface of each pair of subunits) and to require the simultaneous binding of at least two ligands for receptor activation (Amin & Weiss, 1996; Karlin, 2002). The high Hill coefficient observed for homomeric GABA_{C} receptors in experiments with muscimol-biotin (Vu et al., 2005) is consistent with such a possibility. Linking two (or more) effectors into a single, multivalent molecule may therefore lead to more potent ligands due to a linkage-induced entropic advantage, and could be critical for meeting the requirement of multiple ligand binding. Multivalent ligands thus represent a potentially important type of effector, and we will prepare a group of such compounds for testing. AchBP is known to form a symmetric pentamer with the overall shape of a barrel having an outer diameter of about 80 Å, an inner diameter of about 16 Å, and a height of about 60 Å. The ligand-binding sites are approximately equatorial and are about 25 Å from the barrel's center (Brejc et al., 2001). Assuming a similar structure for the GABA_{C} receptor suggests two possible modes of binding for a pair of effectors (adjacent sites vs. nonadjacent sites) and two ways of connecting them (through the center of the protein or around its circumference) (FIGS. 20 and 21). However, as access to the ligand-binding sites of AchBP is thought to be from the outside (Brejc et al., 2001), it is likely that a linker would go around the outside of the GABA_{C} receptor. These considerations suggest that the linker must minimally be 80 Å long and could need to be as long as 130 Å. Distances this long cannot readily be spanned by a hydrocarbon linker in water because the requisite chain length would make the molecules insoluble. Our primary choice of linker is therefore PEG, which is highly flexible and water-soluble. PEG has an effective length of 0.7-2.8 Å per EG unit, and thus the length of PEG linkers that must be considered is about 30-50 EG units (molecular weights: 1300-2200). Monodisperse PEG of n=28 is commercially available (Polypure; Oslo, Norway); the monomer and dimer of this product, together with the commercial availability of a wide variety of PEGylating reagents, thus afford reasonable coverage of the desired=30-50 EG unit range. Dimers will be prepared of azobenzene photoswitches conjugated with suitable muscimol- and phosphinate-based compounds identified as described above, and the PEG length requirement will be tested systematically. FIG. 20 depicts the design of PEG-linked bivalent effectors. Dotted lines in A-B depict boundaries of the pentameric GABA_{C}; open circles are effector binding sites; closed circles are effectors; and curved lines are PEG chains. Possible binding modes employ adjacent (A) or non-adjacent (B) sites. Linker length estimates assume 15 Å from the binding site to the receptor circumference and 2.8 Å per EG unit. C-D show free (C) and tethered (D) forms of the bivalent ligand. Filled circles in C represent effectors; filled ovals in D represent effectors or effector/photoswitch assemblies. FIG. 21 depicts a synthetic route to Y-shaped PEG-linked effectors (filled circles in FIG. 20) or effector/photoswitch assemblies (filled ovals). U-shaped molecules (not shown) containing, e.g., muscimol as effector will be prepared by the reaction of muscimol with bifunctional PEG-bis(NHS ester) reagents.

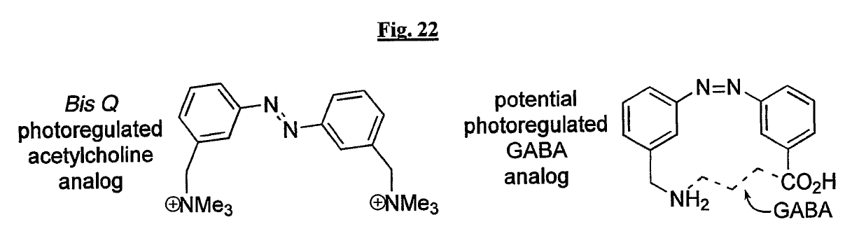

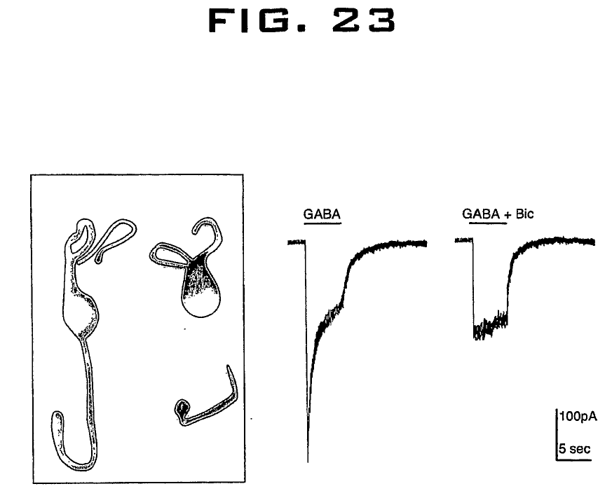

(3) Photoaffinity attachment of effector/photoswitch/linker: The above strategies 1-2 emphasize the importance of determining the distance between the GABA_{C} ligand-binding site and the receptor site at which the distal end of the linker ultimately will be anchored. Photoaffinity labeling is used to covalently attach a suitable peptide anchor at a specific GABA_{C} site. However, it is conceivable that an effector/photoswitch/linker assembly incorporating a distal photoaffinity probe (i.e., lacking a peptide anchor) could exhibit covalent, photoisomerization-dependent attachment at a specific GABA_{C} site upon photoaffinity linking illumination, with the site specificity conferred by the combination of (i) effector binding at the ligand site, (ii) the isomeric state of the photoswitch moiety, and (ii) the length of the linker. This alternative approach of bypassing the need for an inherently site-selective anchor has a low probability of yielding a physiologically functional device, in large part because it is unlikely that features (i-iii) in themselves can establish the desired anchoring specificity. However, this strategy is available in the event of difficulties with the primary approaches. Importantly, it can potentially serve as a molecular “yardstick” for mapping the attachment site(s) of an assembly with given linker length. Additional alternative approaches are: (4) Photoswitch analog of GABA: Here, the azobenzene nucleus would be inserted into the GABA backbone. The rationale is that azobenzene can position amino and carboxyl substituents on neighboring rings at distances comparable with that of the respective groups in GABA (FIG. 22). FIG. 22 shows left: Bis Q, a known photoregulated nAchR agonist in its active, trans form. Right: Proposed azobenzene-based GABA analog in cis form with GABA backbone (dashed bonds) superimposed. While it remains to be determined whether the GABA_{C} ligand-binding site can accommodate such a large template, Lester et al. (1986) found that an analogous compound (“Bis Q”) containing two choline-like side chains exhibits agonist activity at nicotinic Ach receptors. Synthesis procedures for the new GABA analog would be analogous to those described for azobenzene-based amino acids synthesized by Ulysse & Chmielewski (1994) and by Park & Standaert (1999, 2001).

Biophysical and electrophysiological testing of GABA_{C} effector interaction: Determining the activity of a given test effector or effector/photoswitch/linker assembly (FIG. 16) will be based on results obtained in electrophysiological experiments (see below), and in cell-based and in vitro experiments measuring binding of the test effector to GABA_{C}-expressing cells and to isolated GABA_{C} protein. This section describes cell-based assays and in vitro reconstitution experiments to determine the strength and specificity of the effector-GABA_{C} interaction. The in vitro reconstitution assays will employ soluble GABA_{C} extracellular domain, and solubilized or membrane-associated full-length protein. The primary preparation to be used for the cell-based binding assays will be GABA_{C}-expressing neuroblastoma cells. The multiple proposed binding assays described below will provide characterizations on which to base conclusions about the effectiveness of a given test effector. In the event that an assay is for some reason unworkable or inconclusive, the availability of multiple assays should still permit meaningful characterization.

Binding affinity and photoaffinity labeling: Determining the GABA_{C}-binding activity of a given test component (free effector or effector/photoswitch/linker) will typically begin with (³H)GABA competition binding assays performed on intact GABA_{C}-expressing cells of the neuroblastoma cell line. The rationale for initial use of this assay is its logistic simplicity; that is, it does not require modification (i.e., radiolabeling) of the test ligand. Here, we will determine the concentration of test ligand required for criterion (e.g., 50%) displacement of bound (³H)GABA from the cells. Candidate ligands identified in this initial test will be further investigated in competition binding assays with isolated GABA_{C} (full-length or extracellular domain). These tests of binding with isolated GABA_{C} will specifically address the possible pitfall, in whole-cell assays, that (³H)GABA uptake or ligand binding at non-GABA_{C} sites (beyond that routinely compensated for through the use of non-GABA_{C}-expressing cells as controls) rather than actual GABA_{C}-specific binding, contributes significantly to the measured level of binding. Candidate ligands identified in competition binding assays may be further used in saturation binding assays with GABA_{C}-expressing cells and isolated GABA_{C}. Here, the agent may be prepared to contain a ³H radiolabel. The saturation binding data will be evaluated (Scatchard analysis; e.g., Kim et al., 1992) to yield values for binding affinity and number of binding sites. Evaluation of the binding parameters determined for different test ligands will yield a ranking of their potential suitability in ultimately assembled platform structures. However, we will consider the possibility that the ranking established by these tests of free ligand might not be fully applicable to predicting its activity when anchored to the receptor.

AFM analysis: Upon the identification of a candidate ligand in the GABA_{C}-binding experiments, we conduct AFM processes similar in general design to those of Saifuddin et al. (2003), to examine the interaction of the ligand with isolated GABA_{C} extracellular domain. The main question to be addressed will be whether GABA_{C} exhibits specific binding affinity for the ligand. To determine specificity, the test agent or, as control, an inactive analog, will be immobilized on a solid support either through a biotin-avidin interaction (Saifuddin et al., 2003) or by chemical cross-linking to the substrate, and surface changes correlated with the introduction of the GABA_{C} protein will be quantitatively analyzed. As a further control, the test ligand will be examined for its interaction with putatively inactive proteins. In particular, AFM will provide information on integrity of the presumed pentameric structure of the GABA_{C} protein. Surface-force measurements: In similar preparations, we use AFM to obtain surface-force data for the interaction of GABA_{C} extracellular domain with test effectors and effector/photoswitch/linker assemblies. Procedures for AFM tip preparation and data collection will follow those described by Schmitt et al. (2000). Such measurements potentially can provide insight into, e.g., the relative strengths of GABA_{C} binding of monovalent vs. multivalent ligands (FIG. 20), and possibly also on structural correlates of the test component/GABA_{C} interaction (e.g., the range of tolerated PEG linker lengths, a consideration important for linker optimization).

Possible pitfalls: Evaluation of the activity of a given test component will be based on combined results obtained from the reconstitution/cell-based binding procedures described above, and from electrophysiological procedures (see below). Compounds found to be electrophysiologically active will exhibit binding activity. However, a possible outcome is that data from reconstitution and cell-based binding procedures indicate activity of a given test ligand at GABA_{C} receptors, but the compound lacks electrophysiological activity. While this obviously would preclude use of the candidate ligand in the final NNP, such a result would be of fundamental pharmacological interest and could provide insight toward further development of the NNP anchor. We will specifically consider the possibility, with in vitro tests of isolated GABA_{C} (full-length or extracellular domain), that observed binding of a test compound could reflect interaction with a site on the protein not accessible in vivo to the extracellularly located compound, and thus could be irrelevant.

Micelle-incorporated test ligand: The aqueous solubilities of the new muscimol and phosphinic acid compounds considered above are as yet unknown, and it is conceivable that the solubility of a given compound might limit the feasibility of its investigation in GABA_{C}-binding or electrophysiological experiments. The consequence of such a problem could be that a candidate compound (i.e., one with possibly high intrinsic activity when incorporated in an anchored platform but not amenable to aqueous delivery as a free compound at the concentrations needed for characterization) is rejected or overlooked. We use sterically stabilized mixed micelles as a solubilizing medium if a candidate ligand under study is found to display solubility problems. Compositions of the micelles to be employed and procedures for their preparation will follow those routinely used for solubilizing hydrophobic drugs such as the potent anti-tumor agent paclitaxel (e.g., Krishnadas et al., 2003). If needed, a similar approach can be undertaken for the delivery of anchors or complete NNP assemblies.

Electrophysiological testing: As primary systems for electrophysiological testing of candidate effectors and other platform components, we use GABA_{C}-expressing Xenopus oocytes and neuroblastoma cells, and native GABA_{C}-expressing bipolar cells isolated from the rat retina. We also inject a given test component into the intact mouse eye (see below). Whole-cell patch recording from both isolated bipolar cells (Qian & Dowling, 1995; Qian et al., 1997) and mammalian cells (see below), is used in these preparations using the requested patch-clamp recording system to be dedicated to the project. Oocyte recording (e.g., Vu et al., 2005), is done on Xenopus oocytes. The multiple preparations to be used as primary systems have complementary advantages. Xenopus oocytes expressing GABA_{C} (and other) receptors are a robust system with several important advantages. These include the size of the cells (˜1 mm diameter) and their relative ease of handling. The large size establishes a large surface area, affording expression of a large population of receptors. Furthermore, oocytes are routinely suitable for recording over periods of several hr. Typically, initial investigation of a given test ligand will utilize the oocyte system. For these and the other electrophysiological experiments involving tests of components that contain isomerizable photoswitches, the isomeric state of the photoswitch will be measured both shortly before and shortly after the experiment.

GABA_{C}-expressing mammalian cell lines will serve as an intermediate system for testing. While these mammalian cells are much smaller than oocytes and ordinarily permit recording for only shorter periods (˜15-30 min), procedures for their expression of defined receptors, as well as overall cell preparation and maintenance methods, are well established. The experiments will focus on use of the GABA_{C} human ρ1-expressing neuroblastoma cell line described in Section C.3. Isolated retinal bipolar cells of the rat will serve as a model system for testing the action of ligands on native GABA_{C} receptors of retinal neurons. Although there is evidence to suggest that native GABA_{C} receptors of rat retinal bipolar cells are heteromeric (composed of ρ1 and ρ2 subunits; Zhang et al., 1995), pharmacological properties of native GABA_{C} receptor activation are very similar to those of the homomeric ρ1 receptor formed in expression systems (Feigenspan et al., 1993; Pan et al., 1995; Zhang et al., 2001).

Preparative procedures: Single, isolated bipolar cells of the rat retina will be prepared using procedures similar to those described for bipolar cells of white perch retina (Qian & Dowling, 1995). These procedures have been successfully used to prepare mammalian (baboon) retinal bipolar cells in culture and to record GABA_{C}-mediated responses (FIG. 23). These cells maintain their native morphology in culture, and three major regions are easily identified: dendrites, which receive inputs from retinal photoreceptors; the cell body; and the axon terminal, which sends output to retinal amacrine/ganglion cells. GABA_{C} receptor-mediated responses have been reported for both dendrite and axon terminal regions of retinal bipolar cells (Qian & Dowling, 1995; Kaneda et al., 2000); GABA receptors present in these distinct cellular regions can separately be activated by local puff (picospritzer) delivery of solutions containing GABA agonist (Qian & Dowling, 1995). As both GABA_{A} and GABA_{C} receptors are present on retinal bipolar cells, pharmacological approaches are used to separate responses mediated by each receptor type. For example, bicuculline will be used specifically to block GABA_{A} activity, and TPMPA will be applied to inhibit GABA_{C}-mediated responses. A given test component (effector alone, or effector/photoswitch/linker) will be examined for both GABA_{C} agonist and antagonist activity, and the potency of observed actions will be quantified by determination of the dose-response relation. Evaluation of the effector's activity and conclusions about its mechanism of action are based also on analysis of the kinetics of effector-elicited responses, and kinetic comparison of these responses with those produced by control compounds including potential contaminants. Performance criteria relevant to the evaluation of a component will be: (1) whether the maximum elicited GABA_{C}-mediated response exceeds 50% of that elicited by GABA; (2) whether the affinity of the component (from dose-response determinations) is compatible with EC₅₀ ranges for workability; and (3) whether the time scale of the response to the (untethered) test component is sufficiently fast (seconds or faster) to afford potential, at least prototype modulation of neuronal activity in the retina.

In FIG. 23, Left, Solitary bipolar cells isolated from baboon retina are shown. In the Middle are GABA (100 μM) elicits a large transient inward current in a baboon bipolar cell held at −60 mV. Right: The transient GABA response is blocked in the presence of bicuculline (200 μM), leaving a more sustained, GABA_{C} receptor-mediated response.

Pilot electroretinographic (ERG) candidate effectors identified in the binding and electrophysiological processes described above will be further examined in pilot ERG procedures involving in vivo intravitreal injection of the test agent into eyes of anesthetized mice. (Hetling & Pepperberg, 1999; Saszik et al., 2002) (Saszik et al., 2002). The effects of defined quantities of test effector on components of the full-field, dark-adapted ERG including the rod photoreceptor-mediated a-wave and inner retinal components (b-wave and oscillatory potentials) may be confirmed in wild type mice (e.g., C57BL/65). These procedures determine whether the test agent is toxic for, or acts nonspecifically on, ERG components such as the leading edge of the rod-mediated a-wave (a component believed not to depend on the activity of GABA_{C} or other postsynaptic receptors; Pattnaik et al., 2000; Picaud et al., 1998). If the test agent is found in acute experiments (up to several hr) to be non-toxic, subsequent experiments will be conducted to determine whether introducing it alters ERG components for which GABA_{C} receptor activity is thought to play a role. For comparison with responses recorded from wildtype mice, these later procedures may employ a recently described mutant mouse strain that lacks GABA_{C} receptors (McCall et al., 2002).

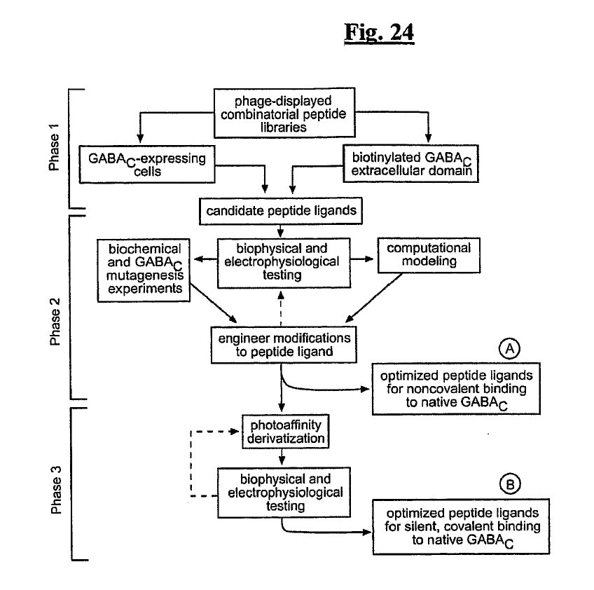

Platform localization/anchoring. Overall organization: NNP operation will require anchoring of the effector-photoswitch complex to the extracellular domain of the GABA_{C} receptor (FIGS. 1 and 16). This section describes the strategies aimed at achieving “silent” (i.e., non-perturbing; see below) covalent attachment of the NNP to the native, i.e., non-mutated, receptor. FIG. 24 diagrams the interrelationship of the approaches proposed to achieve this goal. These will be based on the use of phage display technology to identify 12-mer peptide ligands that display high affinity for the GABA_{C} extracellular domain, and proceeds in three phases. Phase I uses two complementary strategies to select peptides with high GABA_{C} binding affinity: cell-based screening, (i.e., screening against intact GABA_{C}-expressing sf9 and neuroblastoma cell lines); and screening in vitro against isolated GABA_{C} extracellular domain. Synthesized peptides with sequences determined through these screening approaches may be tested in biophysical/electrophysiological assays to identify “first-generation” peptide anchors. Phase 2: A combination of approaches may optimize the peptide's noncovalent binding to the native receptor. This engineering of modifications to the peptide ligand will be based on results obtained from mutagenesis/biochemical experiments and from computational modeling. Recursive engineering and biophysical/electrophysiological testing (cf. upward dashed arrow within Phase 2) will yield determination of the sequences, binding affinities, and sites of noncovalent binding to the native receptor (i.e., GABA_{C} amino acid position) of these optimized “second-generation” peptides (box “A”). Phase 3: Phase 2 optimized peptides are checked for photoaffinity derivatization, covalent (photoaffinity) attachment to native GABA_{C}, and biophysical/electrophysiological testing of the peptide-receptor conjugate. The objective is to identify those peptides whose covalent attachment to native GABA_{C} preserves normal receptor function (“silent attachment”) and, for each of these peptides, the GABA_{C} amino acid position of photoaffinity attachment (potentially, a single site determined by the noncovalent interaction of the parent peptide with the receptor) (box “B”). The FIG. 24 plan is analogous to paradigms used in pharmaceutical drug design. That is, an economical approach (here, phage display) is used with the known target (GABA_{C}) to obtain as many initial “hits” (candidate peptide sequences) as possible. Based on optimization, the number of candidate sequences is reduced, or “filtered”, so that labor-intensive further investigation (Phase 3 photoaffinity tagging and analysis) is carried out only on the most promising candidates. In FIG. 24 dashed arrows denote the “feedback” of results obtained, which will guide the optimization experiments of Phases 2-3.

Phase 1: Phage-display identification candidate peptide ligands. Phage display technology is well suited for the present goal of obtaining peptide ligands that interact selectively and tightly with the target receptor's extracellular domain. In phage-display, combinatorial peptides are expressed at the amino-terminus of protein III on the surface of bacteriophage M13, encoded by degenerate oligonucleotides of fixed length. Phage display offers the advantages that: (1) the peptides expressed on the surface of the viral particles are accessible for interactions with their targets; (2) the recombinant viral particles are stable (i.e., can be frozen, exposed to pH extremes); (3) the viruses can be amplified; and (4) each viral particle contains the DNA encoding the recombinant genome (Kay et al., 1996). Consequently, these libraries can be screened by isolating viral particles that bind to targets, plaque-purifying the recovered phage, and sequencing the phage DNA. Phage-displayed combinatorial peptide libraries have proven useful in identifying novel ligands for membrane receptors and other proteins [e.g., Johnson et al., 1998; Paige et al., 1999; Kay et al., 2000; Sidhu et al., 2003]. Over the past 12 years, peptide ligands to over 30 different protein targets have been isolated, including the ectodomain of the herpes virus entry mediator A, a member of the tumor necrosis factor receptor family (Sarrias et al., 1999). Peptide ligands for the GABA_{C} receptor may be identified as well.

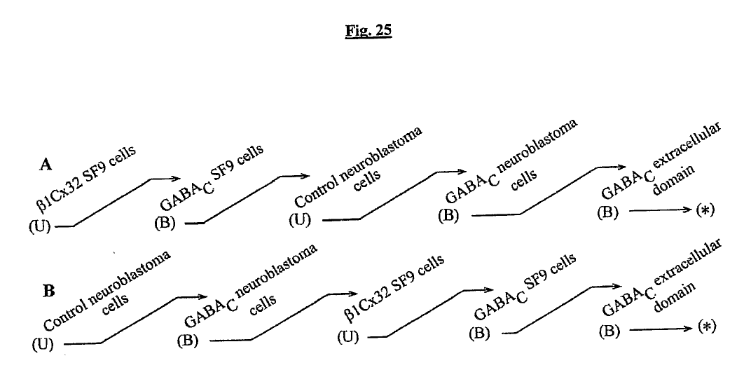

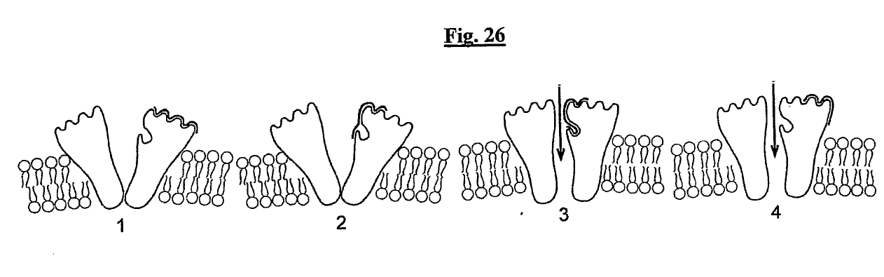

FIG. 25 shows strategies A and B for phage screening. Symbols B and U denote, respectively, the selective recovery of bound and unbound phage particles. Asterisks denote populations of phage in the final output.

Cell-based phage screening: Using a large collection of phage-displayed combinatorial peptide libraries from the Kay lab, we will use a cell panning procedure to select phages that specifically bind to GABA_{C}-expressing cells. As the cells will express many proteins in addition to the expressed GABA_{C} that can bind the phage, we will use a “ping-ponging” approach with two different cell types (neuroblastoma cells and baculovirus-transfected insect cells) to isolate GABA_{C}-binding phage (FIG. 25). This strategy, which assumes that the only common cell surface protein will be GABA_{C}, has been used successfully in previous studies (Goodson et al., 1994). We anticipate that this screening procedure, following multiple rounds of biopanning, will upon sequencing of the phage's inserts encoding the 12-mer expressed peptide yield candidate 12-mer peptides with specific GABA_{C}-binding activity. Support for this comes from preliminary data (section C.5), which suggest that peptide ligands can be isolated from a phage-displayed combinatorial peptide library screened against GABA_{C}-expressing neuroblastoma cells. Immunofluorescence tests on GABA_{C}-expressing cells will serve as a further assay for binding activity. If the number of differing phage sequences resulting from the above-described screening procedure is very large, we will use a whole-phage binding assay (Heitner et al., 2001) to confirm binding of the phage to intact cell surfaces. Phage particles from individual clones expressing the putative peptide ligand will be incubated with GABA_{C}-expressing neuroblastoma cells and with control, non-GABA_{C}-expressing neuroblastoma cells. Following washing steps to remove unbound phage, the cells will be incubated with a mouse monoclonal antibody to the M13 phage (Amersham Pharmacia) (Maruta et al., 2002) and then with FITC-conjugated secondary antibody. If the phages expressing the candidate peptide specifically bind to GABA_{C} receptors, the fluorescence signal measured for the GABA_{C}-expressing cells treated with test phage should exceed the fluorescence signal of the controls. For phage that have been confirmed to bind to cells expressing GABA_{C}, biotinylated forms of the peptides will be synthesized and used for co-localization studies using fluorescently labeled streptavidin (Molecular Probes) to detect the bound peptide. A rabbit polyclonal antibody to the intracellular loop of GABA_{C} receptor (Santa Cruz Biotechnology) may be generated by conventional methods (Hanley et al., 1999), affinity purified and used, together with a different, fluorescently labeled secondary antibody, for detection of the receptor. Co-localization are determined by confocal microscopy (Leica DM-IRE2 microscope housed in the UIC Dept. of Opthalmology and Visual Sciences Core facility). Initially, GABA_{C}-transfected neuroblastoma cells are used with non-transfected cells as controls. Cells are fixed with 4% formaldehyde and permeabilized, and varying concentrations of primary antibody, peptides and secondary reagents are used to optimize the signal/background ratio. To determine if the peptide remains attached to the cells during the fixation and subsequent steps, we compare the signal obtained from unfixed cells following sequential incubation with the peptide and labeled streptavidin with the signal obtained from cells fixed, permeabilized and similarly treated.

In vitro screening against isolated extracellular domain: Biotinylated protein targets will be used for in vitro screening of phage-displayed combinatorial peptide libraries. Purified GABA_{C} extracellular domain obtained using the bacterial or baculovirus expression system are chemically biotinylated with the Pierce Biotinylation kit to attach biotin to the ε-NH₂ of lysine residues within the target protein. Since there are multiple lysines in the GABA_{C} extracellular domain (10 for human ρ1; 9 for perch ρ1B), and one or more may be important for functional binding of GABA, partial biotinylation conditions are used so that only 1-2 lysines are modified on average. To test for functionality of the modified form, we perform binding assays on the biotinylated material before and after immobilization with streptavidin-coated surfaces, and determine whether the target protein is still active. Approximately 200 μg of biotinylated protein are needed to select phage and confirm binders. For selection, the biotinylated protein are incubated with super-paramagnetic, polystyrene beads that have streptavidin covalently attached to their surface. We screen 23 different libraries for peptide ligands to the GABA_{C} target. These libraries consist of 12-mer combinatorial peptides, with fixed amino acids such as cysteine at various positions within the peptide. It is noteworthy that since bacteriophage M13 is secreted from bacteria, peptides with multiple cysteines will form intramolecular disulfide bonds, often yielding strong binding ligands (Yamabhai et al., 1998). Phage ligands from most of these libraries (Scholle et al., 2005) and other similar libraries have been isolated. After three rounds of affinity selection, a phage-based ELISA will be used to quantify phage binding to the biotinylated target compared to negative control proteins such as bovine serum albumin, SH3 domains, streptavidin, and other biotinylated proteins. Liquid handling robotic workstations (Beckman FX robot, plate washers, etc.) may be used for the high-throughput processing of libraries.

Biophysical/electrophysiological testing: Peptides determined from screening with whole cells and isolated extracellular domain, henceforth termed “phage-derived peptides”, are synthesized. Following initial optimization of the peptide sequence through systematic residue replacement and analysis of in vitro binding affinities (see below), candidate peptides are supplied to the other Investigators for tests of binding activity through assays. The nominally desired activity of the peptide(s) being sought is a physiologically “silent” (i.e., non-agonist, non-antagonist) attachment at a site on the GABA_{C} extracellular domain distinct from the GABA-binding site (FIG. 16; and FIG. 26, panel 1). However, alternative activities are possible (panels 2-4), and unanticipated activities could prove to be interesting (panels 2-4). As the nominally desired silent peptide will itself lack electrophysiological activity, the characterization of GABA_{C} binding of candidate peptides will require a “toolbox” of assays. The sections below describe procedures that will be available for initial optimization and characterization of a given candidate peptide. We anticipate that not all of the testing procedures will be applied to every first-generation peptide; as the research proceeds, results obtained on the logistic efficiency and stringency of a given assay will govern decisions as to its use in later experiments.

FIG. 26 depicts possible interactions of phage-derived peptide (thick wavy line) with the GABA_{C} receptor (for simplicity, shown here as a two-subunit receptor as in FIG. 1). 1: “Silent” binding at a site distinct from the receptor's ligand-binding site (nominally desired interaction). 2: Inhibitory interaction (blockage of ligand-binding by the receptor). 3 and 4: Activating interactions in which the peptide mimics GABA (3) or acts allosterically (4).

Initial optimization of critical residues in peptide ligands: Using results obtained from the two phage screening approaches and initial biophysical/electrophysiological testing, we sharpen the definition of the ligand preferences with chemically synthesized peptides. Here, peptide synthesis are necessary, for several reasons. First, certain peptide sequences may be absent from the library because they interfere with viral morphogenesis or secretion. It has been observed that peptides with runs of arginines (Peters et al., 1994) or odd numbers of cysteines (Kay et al., 1993) are not displayed efficiently on bacteriophage M13. Second, sometimes only a small number of binding isolates are recovered from phage-display experiments, making it difficult to recognize a consensus. Third, because the peptides are displayed on protein III, which is pentavalent on M13, it is difficult to discriminate between weak and strong binding interactions due to avidity effects, i.e., multivalent interactions between phage and the immobilized target. Thus, it is hard to know how to weight the contributions of residues that vary between phage-displayed peptides toward binding. Initially, using small-scale syntheses, we prepare peptides that have been truncated at the N- or C-terminus to determine the boundaries of the peptide's binding element, and in which residues have been systematically replaced with alanine (Yamabhai & Kay, 2001) to determine which residues contribute most to binding. An Advanced ChemTech Apex 396 robot may be used to synthesize via standard Fmoc chemistry (Merrifield, 1965) up to 96 peptides at a time, in small scale (<1 mg). Their N-termini are chemically biotinylated, and binding of the resulting peptides will be determined in vitro by an enzyme-linked assay (binding to immobilized target monitored using streptavidin conjugated to alkaline phosphatase). Once critical positions have been defined, they may be replaced with other amino acids to see if this replacement improves binding. Often, the binding of phage-derived peptide ligands to their targets can be improved 3-5 fold by systematic residue replacement/optimization (DeLano et al., 2000).

Binding affinities and binding kinetics of peptide ligands: We synthesize the selected peptide ligands on a larger scale (˜10 mg or greater), and determine their GABA_{C}-binding properties by isothermal titration calorimetry (ITC) and by in vitro/whole-cell assays (see below). These larger-scale syntheses also employ the Advanced ChemTech Apex 396 instrument. The peptides will be HPLC-purified and their quality will be evaluated by MALDI-TOF mass spectrometry. The dissociation constant for the binding of a given peptide to the GABA_{C} extracellular domain will be measured by ITC. ITC affords determination of the separate contributions of changes in enthalpy (AH; typically indicating changes in electrostatic, van der Waals and hydrogen-bond interactions) and entropy (ΔS; typically reflecting changes in solvation entropy and conformational entropy) to equilibrium binding, as well as the value of the equilibrium binding constant (e.g., Leavitt & Freire, 2001). It thus can provide important insight into the molecular mechanism of the binding reaction. Suppose, for example, that ITC measurements for a given candidate peptide's binding to GABA_{C} suggested the change in ΔS to be the dominant factor driving the binding reaction. This result would suggest the possibility that a hydrophobicity-increasing modification of the peptide's sequence would produce even tighter binding to the receptor, and would accordingly motivate undertaking preparation/testing of such a modified peptide. Dissociation constants (K_{D}'S) of peptides recovered by phage display, when synthesized and tested in solution, typically range from 10 μM to 300 nM (Hyde-DeRuscher et al., 2001), and occurrence of a K_{D}˜10⁻⁶ M or lower will serve as a key performance criterion for further investigation of a given peptide. From the dissociation constant K_{D}, one can estimate the k_{dissoc}, the dissociation rate constant (in s⁻¹), through the relations, K_{D}=(k_{dissoc})(k_{assoc})⁻¹, and (k_{dissoc})[peptide-GABA_{C}]=(k_{assoc})[peptide][GABA_{C}], that describe the association of peptide and GABA_{C} to form a complex, where k_{assoc} (in M⁻¹ s⁻¹) is the association rate constant. Assuming k_{assoc}˜10⁸ M⁻¹ s⁻¹ as a diffusional association rate, setting (for illustration) K_{D}=1 μM yields k_{dissoc}˜10² s⁻¹, i.e., ˜10 ms for the dwell time of the noncovalently bound peptide. Beyond emphasizing the ultimate need for covalent peptide attachment, this estimate might seem problematic for the Phases 1-2 objectives of identifying/characterizing noncovalently bound peptides. Importantly, however, peptide synthesis on the large scale will permit driving of the association reaction, by sufficiently high concentrations of peptide, to render workable the measurements of (instantaneous, equilibrium) noncovalently associated peptide-GABA_{C}. Furthermore, strategies are available for increasing the stability of the peptide-GABA_{C} interaction, thus reducing k_{dissoc}. One such strategy will be to create divalent or multivalent forms of the peptides which, through the phenomenon of avidity, will exhibit greatly enhanced binding to the pentameric receptor (Mourez et al., 2001). Another will be to select, for GABA_{C} binding, human single-chain Fragments of variable regions (scFv's) from a phage library; scFv's tend to bind to targets with low nanomolar K_{D}'s due to their stable three-dimensional structure (Sheets et al., 1998).

GABA_{C}-binding assays and AFM experiments: We determine the strength of binding of candidate peptides to GABA_{C}-expressing cells (e.g., neuroblastoma cells) and isolated extracellular domain/full-length GABA_{C}. In these binding experiments, which will involve the synthesis of radiolabeled peptide ligand, we will consider the possibility that the state of the GABA_{C} receptor (open or closed) influences peptide binding, as has been observed for certain ligands in other receptor systems (e.g., Djellas et al., 1998). (The nominal objective in the present project is state-independent binding of the peptide.) This possibility will be tested by determining whether added GABA (and thus, occupation of the receptor's ligand sites) alters binding of the radiolabeled peptide. AFM processes will test the specificity of GABA_{C}'s binding of a given test peptide. Here, with surface tethering of the candidate peptide vs. (as a control in separate preparations) a known nonreactive peptide, and with use of isolated GABA_{C} extracellular domain, we characterize the GABA_{C}-peptide interaction. Of particular interest here will be the dependence of the interaction on the peptide site (amino acid position) used for tethering, and on the surface density of the tethered peptide. The AFM data will provide insight into the mode of peptide conjugation to NNP effector, photoswitch and linker components that will preserve the peptide's GABA_{C}-binding activity.

Electrophysiology: Candidate peptide sequences may be tested for GABA_{C} activity electrophysiologically. Electrophysiology will not be a stringent test of the peptide's activity, i.e., peptide binding to the GABA_{C} extracellular domain may be silent. A peptide may have agonist activity (FIG. 26, panel 3), and that peptide may be an effector moiety. We do not anticipate difficulty in preparing candidate peptides in amounts needed for electrophysiological testing. That is, taking the molecular weight of the 12-mer peptide as ˜1,000 Da, the preparation of ˜1-10 mg of peptide (which is straightforward) will yield several ml of a 1 mM solution, a concentration far exceeding the ˜10 μM upper limit of the anticipated K_{D} of the peptide.

Binding in retinal tissue: The binding of candidate peptides to GABA_{C} receptors of retinal bipolar cells will be analyzed also in immunofluorescence experiments. Frozen cryosections (16 μm thick) from mouse retina will be mounted on polylysine coated slides and incubated with biotin-labeled peptide and antibodies to GABA_{C} receptor. A biotin-labeled control peptide that does not bind to retina will be used to assess binding specificity. Bound peptide and primary antibody will be detected by fluorescently labeled streptavidin and secondary antibody, respectively. The receptor specificity of the candidate peptide anchor may be determined by comparing the GABA_{C} co-localization signal with that obtained for a differing expressed receptor, e.g., GABA_{A} α₁β₂γ₂ receptors. Such specificity of receptor binding will be critical for functionality of the ultimately envisioned NNP (FIG. 1), and the screening procedure to be used in the present experiments (FIG. 18) is intended to yield GABA_{C} specificity. However, cross-reactivity of a given candidate peptide with, e.g., the GABA_{A} receptor need not be a major setback to achieving platform anchoring in GABA_{C}-expressing model cells.

Optimization of noncovalent peptide binding: By recursive biophysical/electrophysiological testing and peptide modification (FIG. 24), we optimize the peptide ligand sequence and obtain functional/structural information on the nature of the peptide-GABA_{C} interaction. The precise atomic details of the peptide-GABA_{C} interaction may determine, e.g., directions in which the peptide chain could be extended/shortened to yield tighter binding to the receptor. NMR spectroscopy and X-ray crystallography of the complex formed by the peptide's noncovalent binding to the GABA_{C} extracellular domain can provide such information. However, NMR analysis will require relatively high concentrations of the target receptor (˜10-20 mg/ml) that can remain properly folded and in solution over the extended period of data collection. Similarly, crystallization will require large amounts of receptor, and the success of crystallization of the complex cannot, of course, be presumed. NMR and crystallization remain potentially attractive approaches. However photoaffinity labeling and GABA_{C} mutagenesis are two analytical-scale approaches that will require orders of magnitude less material than NMR or crystal studies of the peptide-GABA_{C} complex. Facilitating these two experimental approaches will be computational modeling of GABA_{C}. In the event of rapid development of methods for preparing well-folded GABA_{C} extracellular domain in quantities needed for NMR and/or crystallization of the complex, processes may be re-directed to emphasize these approaches.

Procedures with engineered GABA_{C}: We use site-directed mutagenesis techniques to introduce a cysteine residue within the extracellular domain to afford covalent anchoring of a given test system (e.g., azobenzene-derivatized effector; through a thiol-reactive moiety such as maleimide that can readily be introduced into the test system. Cysteine substitution has been widely used to probe structure-function relationships of proteins including, for example, the GABA-binding pocket and channel lining domain of GABA receptors (Xu & Akabas, 1993; Chang & Weiss, 2002; Newell & Czajkowski, 2003). The method is commonly used as a substituted-cysteine accessibility assay, where the accessibility of a native amino acid residue participating in a particular function of the protein is inferred from accessibility of the introduced cysteine to sulfhydryl group modification (Karlin & Akabas, 1998). By contrast, the present use of cysteine substitution involves selection of an amino acid position on the GABA_{C} extracellular face that is not essential for receptor function, analogous to the approach employed by Banghart et al. (2004). Thus, linkage of an NNP to the introduced cysteine residue preserves the native GABA_{C} receptor's functionality (ligand-gating of the chloride channel). The selection of initial GABA_{C} amino acid sites for substitution will be based on previous indications that for GABA_{A} receptor subunits, introducing a foreign tag between the fourth and fifth amino acid after the signal peptide yields expression of the tag sequence at the receptor surface with preservation of receptor function (Connolly et al., 1996). Preliminary results indicate such a property of GABA_{C} receptor ρ subunits. Introduction of a cysteine at this location in GABA_{C} thus will likely yield an exposed sulfhydryl group on the receptor surface. The selection of candidate receptor sites for further investigation by cysteine substitution may be based on photoaffinity labeling data and computational modeling results (see below), as well as on results from the initial cysteine substitution procedures. For a given site of mutagenesis, the effect of cysteine substitution at the selected position is first tested in electrophysiological/binding experiments on unconjugated receptor, vs. receptor incubated with a sulfhydryl-specific florescent reagent such as TEXAS RED™-MTSEA (Toronto Research Chemicals). If these initial procedures indicate both preserved function of the receptor and accessibility of the cysteine, one may proceed to investigate peptide ligands that have been modified to contain a thiol-reactive moiety. The objective here is tethering the peptide to the cysteine-substituted receptor in permanent fashion.

Photoaffinity labeling for covalent anchoring to native receptor: Peptide ligands modified through conventional methods to incorporate a photoaffinity probe may be used on isolated GABA_{C} extracellular domain and on GABA_{C}-expressing cells, to map the amino acid positions of native GABA_{C} at which candidate peptide ligands bind (FIG. 24). In vitro experiments on photoaffinity mapping traditionally have employed a radiolabel photoaffinity probe, digestion of the tagged protein target with proteases, and purification/identification of the modified (radiolabeled) amino acids of the target. However, current mass spectrometric (MS) methods suitable for protein analysis now often permit a non-radiolabel approach; modified regions of the protein are identified by changes in HPLC retention times of tryptic fragments, and specific labeled residues are identified by MS and microsequencing of the tryptic fragments. There are four major classes of photoaffinity probes: aryl azides, benzophenones, diazirines, and α-diazocarbonyl compounds, each of which has advantages and disadvantages. Our initial approach, based on past experience (e.g., Turek et al., 2002) and the commercial availability of a wide spectrum of reagents, will be use of the aryl azide, a probe that is activated by light of ˜260 nm wavelength. (However, fully assembled NNPs containing muscimol as an effector moiety will require use of a photoaffinity probe that absorbs at longer wavelengths, as muscimol is photolabile at wavelengths near this value. The position for incorporation of the photoaffinity probe is at the N- or C-terminus of the peptide. The core of the 12-mer peptide largely mediates the interaction with the receptor, and that the termini are not within a surface groove and thus likely of relatively little importance to binding. The peptide can be modified through its N-terminal amino group using an appropriate linker reagent. Alternatively, the peptide can be re-synthesized to incorporate an Fmoc benzophenone photoaffinity probe at any position (also cf. Bosse et al., 1993; Tian et al., 2004). For example, successful crosslinking of azidophenylalanine-modified insulin to the insulin receptor has been reported (Kurose et al., 1994). We use alanine scanning to identify candidate sites for incorporation of the photoaffinity probe. Peptide positions for which alanine preserves receptor binding affinity will be interpreted as positions that do not contribute directly to binding and thus are candidates for benzophenone incorporation. GABA_{C}-expressing cells: The primary photoaffinity approach involves in vitro testing, i.e., the use of isolated GABA_{C} extracellular domain. However, to test the validity of the in vitro results obtained, we also map the site of target protein tagging on GABA_{C}-expressing cells. This necessarily more complex type of process, outlined in the following four-step procedure, will be performed only for peptides that appear promising based on the in vitro results. (1) Preparation of photoaffinity-tagged and biotinylated peptide (here termed peptide PB): The test peptide is derivatized to incorporate a biotin moiety (e.g., at the peptide's N-terminal) and a photoaffinity agent. Competition binding and electrophysiological assays of peptide PB's activity, as well as pull-down assays similar to those previously used (Nielsen & Kumar, 2003), will be conducted to determine if PB retains the activity of the parent underivatized peptide. We then further derivatize the biotinylated peptide to contain an aryl azide probe at a suitable site. Alternatively, we can employ a commercially available bifunctional probe such as Sulfo-SBED (Pierce) that incorporates both biotin and an aryl azide and can be attached to (cysteine-free) peptide at the N-terminus. (2) Linking illumination: The candidate peptide PB will be incubated with GABA_{C}-expressing cells in the presence (or, as control, absence) of UV (i.e., photoaffinity linking) illumination. This illumination covalently couples (some of) the GABA_{C}/PB complexes present; it also is expected to covalently link PB with other, unwanted target proteins. (3) Recovery of GABA_{C}-PB conjugate: The treated cells are extensively washed to remove unreacted peptide and the cell membranes will be solubilized with CHAPS. The solubilized membranes containing GABA_{C}-PB (and other PB-containing) conjugates are subjected to one of two procedures designed to isolate GABA_{C}-containing material (GABA_{C}-PB conjugate and free GABA_{C}): either immunoaffinity chromatography using anti-GABA_{C} antibody as the immobilizing agent, or ligand affinity chromatography using tethered muscimol as the immobilizing agent. Using streptavidin-coated beads, we then selectively immunoprecipitate the GABA_{C}-PB conjugate and determine its purity by SDS-PAGE and Western blotting. (4) Generation/analysis of PB-tagged GABA_{C} fragment: To determine the site (i.e., local GABA_{C} sequence) at which the peptide PB is bound, we perform limited proteolysis of the GABA_{C}-PB conjugate. This limited proteolysis involves incubation with trypsin or another protease under conditions designed to avoid hydrolysis of the peptide PB moiety of the conjugate. As the PB sequence will be known, PB's preservation during this step can readily be checked. Following purification of the GABA_{C}-PB conjugate by streptavidin-coated beads, we will analyze the conjugate by MS and microsequencing. As the GABA_{C} amino acid sequence is known and peptide PB's sequence will be known, this should yield identification of the GABA_{C} amino acid position photoaffinity-tagged by peptide PB. Limitations/possible pitfalls: While the multi-step procedure just described is likely to have a relatively low overall yield, it should be possible with sufficient scale-up of the starting preparations (including, e.g., a population of GABA_{C}-expressing neuroblastoma cells) to achieve an absolute yield sufficient for MS/microsequencing. Alternatively, photoaffinity experiments may instead employ radiolabeled (rather than biotinylated) affinity-tagged peptide, with corresponding procedures to recover/analyze the radiolabeled conjugate of peptide and GABA_{C} fragment. This approach, however, would require HPLC separation of the digested receptor fragments using a radiochemical detector.

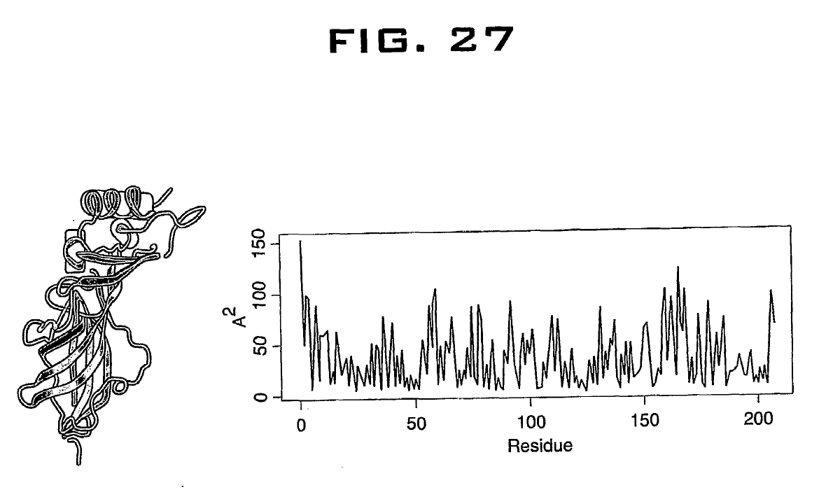

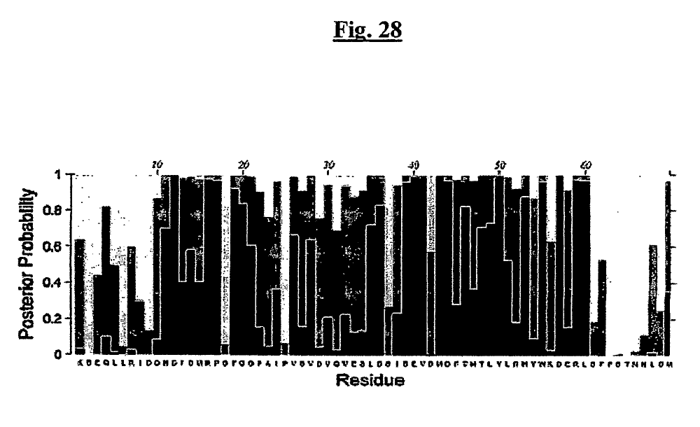

FIG. 27 shows the N-terminal region of AchBP, which will serve as a template for the modeling of the corresponding region of GABA_{C}. The model obtained from Protein Data Bank. The C-terminus of this region is at the bottom. On the right are predicted solvent-accessible surface areas (in square Angstroms; A²) for the N-terminal domain of the human ρ1 GABA_{C} sequence. Peaks indicate amino acid positions predicted to be relatively exposed to the extracellular medium.

Computational modeling: To facilitate the interpretation of data obtained in the photoaffinity and cysteine mutagenesis experiments of Phase 2, and to guide the design of subsequent experiments aimed at optimizing the sites of peptide anchoring, we carry out a two-pronged approach to model both the molecular structure of the GABA_{C} extracellular domain and its evolutionary history. Structural model of the GABA_{C} extracellular domain: We first construct explicitly a homology model structure of the extracellular domain of GABA_{C}. This is based on an AchBP template structure (FIG. 27) and a high quality multiple sequence alignment obtained using psi-blast and culstalW (Altschul et al., 1997; Chema et al., 2003). We use the MODELLER package to build the three dimensional structure (Fiser & Sali, 2003), an approach similar that described by Ernst et al. (2003), and calculate surface-accessible regions on this model structure. To improve our confidence in predicted surface residues, we further predict solvent-accessible surface residues from the GABA_{C} primary sequence using neural network and profile-based techniques (Ahmad & Gromiha, 2003; Gianese et al., 2003) (FIG. 27). Results from the two approaches are compared and consensus regions identified. Because our goal is to locate residue sites that are accessible for cysteine substitution and peptide attachment that will not perturb receptor physiology, the most relevant information sought from this model is identification of the set of surface exposed residues, which will be combined with information obtained from evolutionary analysis. Predictions regarding the spatial conformations of side chains of buried residues will be less important. Amino acid sites predicted to be favorable for, e.g., cysteine substitution, will be chosen from surface residues that are distant from the effector site but are within or adjacent to solvent-exposed patches. To identify energetically most favorable sites, we will generate and analyze an exhaustive set of candidate surface patches using a geometrically confined breadth-first search method (Cormen et al., 2001). Identification of candidate sites of receptor modification: To select candidate GABA_{C} surface sites, we extract information from the evolutionary history of the GABA_{C} receptor. Specifically, we carry out an extensive maximum likelihood analysis using a continuous-time Markov model to estimate the mutation rates at different residues, based on the phylogenetic tree for a set of orthologs and paralogs of the extracellular domain. Through this analysis we will identify amino acid residues that are relatively variable (i.e., not highly conserved) and thus are potential sites of peptide attachment. The continuous-time Markov model and maximum likelihood approach clarifies a controversy in the field of protein folding: namely, whether folding nuclei residues are conserved by evolution (Tseng & Liang, 2004). We have carried out a preliminary study of human ρ1 GABA_{C} and have identified 25 DNA sequences for detailed phylogeny analysis. FIG. 28 shows a preliminary posterior probability analysis of amino acid residues 1-70 of the investigated sequences (data obtained from GeneBank) in which the occurrence among species of synonymous vs. nonsynonymous codon substitutions (Tseng & Liang, 2004) yields a predicted index of mutation rates. Here, darkly shaded, medium shaded and lightly shaded vertical segments at a given amino acid position (whose amplitudes sum to unity) represent, respectively, predictions of relatively high, medium and low conservation. For example, a relatively large amplitude of light shading indicates relatively little amino acid conservation (i.e., high variability) and thus relative likelihood of solvent exposure and accessibility to modification. The illustrated (aligned) sequence is that of human ρ1 GABA_{C}. In addition to predictions of positions for modification, this and related computational analyses (Li et al., 2004; Li & Liang, 2005) will yield predictions for amino acids at subunit interfaces of the pentameric GABA_{C} receptor, a property important to understanding receptor subunit assembly and physiology (Qian & Ripps, 1999). Importantly, the computational modeling approaches just described are necessary even if a crystal structure of the GABA_{C} receptor is achieved. For example, the crystal structure of AchBP is available (Brejc et al., 2001), but does not afford specific predictions for the binding of peptide ligands to be examined. Determination of the GABA_{C} crystal structure is of course desirable and would place the modeling on a firmer foundation, e.g., would allow more accurate determinations of parameters such as solvent accessibility, but would not replace the need for modeling.

Phase 3: Silent, covalent peptide binding to native receptor: We identify from the “filtered” set of candidates (FIG. 24), photoaffinity-derivatized peptides that bind to GABA_{C} in a manner that does not perturb receptor physiology. While this objective of non-perturbative binding is clearly more stringent than the Phase 2 goal of using photoaffinity tagging to map the GABA_{C} position of ligand attachment, we these silent ligands may be a sub-set of, or closely related to, those investigated in the course of Phase 2. Importantly, the identification of silently binding peptides and their sites of photoaffinity attachment is likely to facilitate later-generation structures employing chemical mechanisms of attachment, e.g., peptide derivatization with an amine-reactive, activated ester group rather than a photoaffinity probe. As with the photoaffinity probe, the specificity of this chemically-based covalent attachment would be governed by the binding specificity of the peptide and the proximity of suitable functional groups on the native receptor.

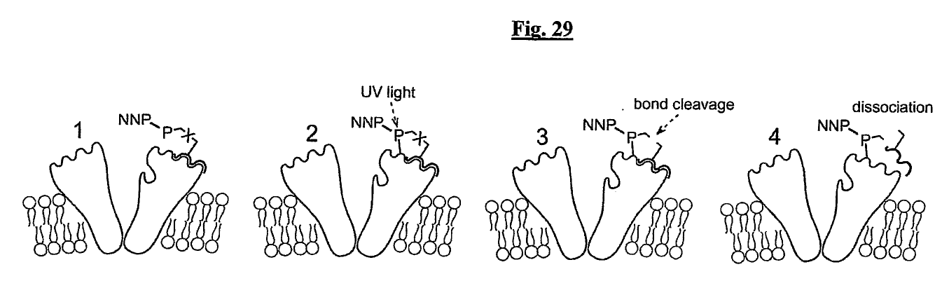

Alternative approaches: (1) If the ultimately isolated peptide ligands lack the desired specificity or binding strength needed for NNP functionality, an antibody substitute may be used. It is possible to display single-chain fragments (scFv's) of antibodies on the surface of phage (Sheets et al. 1998). Advantages of scFv's are that they have a stable three-dimensional structure, often exhibit very high affinity (low nanomolar dissociation constants) for their targets, and can adopt a concave or convex surface to bind target proteins. Antibody fragments to a wide variety of targets have been generated (Han et al., 2004). (2) In the event of difficulties with use of the GABA_{C} extracellular domain for in vitro phage screening, e.g., if the proper folding of GABA_{C} requires membrane insertion, detergent-solubilized full-length GABA_{C} prepared using the baculovirus system may be used. Importantly, the presence of solubilizing detergent such as CHAPS is not expected to interfere with the capacity of phage binding. Here a possible pitfall is the selection of peptide ligands (or scFv's) that are reactive with the cytoplasmic or trans-membrane domains of the receptor rather than the extracellular domain. Results obtained by testing peptide binding on whole GABA_{C}-expressing cells (see section above) allow the exclusion of such peptides as candidates and the focus, in further investigation, on those peptides that exhibit high affinity for cell-expressed GABA_{C} as well as GABA_{C} extracellular domain. (3) A further alternative strategy for achieving (ultimately silent) photoaffinity-mediated anchoring is the use of a scaffold, i.e., a temporary molecular structure, e.g., a phage-derived peptide or chain-derivatized agonist or antagonist that ultimately dissociates from the receptor, to localize the site of binding of a photoaffinity probe that will serve as a covalent anchor (FIG. 29). Here, a cleavable bond (e.g., the phosphate of a hemiacetal that in the presence of endogenous/added phosphatase yields a spontaneously hydrolyzing hemiacetal) initially links the test NNP structure and a photoaffinity probe to the scaffold. Subsequent photoaffinity labeling and scaffold dissociation would establish covalent NNP binding at a site determined by the scaffold's binding. Synthetic peptides related to α-conotoxins (antagonists at neuronal nicotinic Ach receptors; e.g., Azam et al., 2005) may be used as a GABA_{C} scaffold. FIG. 29 depicts the scaffold approach using, as illustration, a noncovalently bound peptide (thick wavy line) as scaffold. The peptide, previously derivatized to incorporate a cleavable bond (X), a photoaffinity probe (P), and other platform components (NNP), attaches to the receptor (panel 1). UV photoaffinity linking illumination (2), chemically induced bond cleavage (3) and peptide dissociation (4) yield the site-directed, covalently bound NNP.

Upon the identification of peptides with high GABA_{C}-binding affinity, it will become important, for refinement of the approaches used, to explore additional measures of the peptide-GABA_{C} interaction. Surface plasmon resonance (SPR): Using SPR, an optical technique that affords time-resolved determinations of binding kinetics, we analyze the interaction of GABA_{C} extracellular domain with a given candidate peptide or, alternatively, with a population of whole phages expressing the peptide. Such SPR determinations for defined peptide sequences, by affording a ranking of these candidate peptide anchors based on kinetic binding parameters, may complement the primary proposed approaches in identifying peptides with high affinity for GABA_{C}. Surface force measurements are taken. These procedures test the interactions of candidate peptides with tethered isolated GABA_{C} and cell-expressed GABA_{C}.

To achieve light-dependent control of GABA_{C} channel gating, we (1) identify a second-generation organic photoswitch whose spectral properties and relaxation kinetics (relative to the unmodified azobenzene photoswitch of effector/photoswitch/linker assemblies) to be tuned to meeting physiological requirements of the ultimate device; and (2) interface effector/photoswitch/linker assemblies with the peptide-based anchors, and biophysical/electrophysiological testing optimizes this interfacing for GABA_{C} control.

Second-generation photoswitches. Modified azobenzenes: The photoconversion of trans to cis azobenzene requires near-UV (366 nm) rather than visible light, and the thermal relaxation of cis to the (favored) trans occurs on a time scale of hours to weeks. Thus, while the slow thermal isomerization of azobenzenes is workable and indeed desirable for the azobenzene-based prototype photoswitches, (as it allows an ample time window for experimental investigation of simple, one-way light-induced changes), meaningful physiological activity of the envisioned structure will require far faster relaxation. In addition, a light-sensitivity of the ultimate, clinically used NNP in the visible rather than near-UV wavelength range is critical, in significant part because the intensity of UV light in conventional environments, and of UV light transmitted by the (native) lens of the eye, is considerably lower than light intensity in the visible (400-700 nm) range. The immediately following paragraphs address these two points.

Photoswitch relaxation time is a critical design parameter for the NNP, as it governs not just how long the GABA receptor remains activated but how fast the device can cycle, i.e., recover sensitivity to an activating photon. The general model of LGIC function includes the concept of an essential locking of bound ligand by the receptor in its channel-open state (Colquhoun, 1999; Bianchi & Macdonald, 2001). In the case of a tethered ligand, the behavior at the binding site is yet to be determined, but for the present discussion we shall assume that the effector moiety of the test system under study behaves as a diffusible ligand. Chang & Weiss (1999) have developed a model of GABA_{C} receptor activation based on a combination of electrophysiology and ligand binding studies on GABA_{C} ρ1 receptors expressed in Xenopus oocytes. This model provides two initial performance criteria for relaxation of the NNP photoswitch. First, the evident transition time to the channel-open state (280 ms; β⁻¹ in Table 1 of Chang & Weiss, 1999) suggests a lower limit of ˜30 ms (˜0.1β⁻¹) for the photoswitch relaxation time, to provide a significant (assumed 10%) probability of channel opening during the lifetime of the photogenerated isomer. (Cis and trans azobenzenes have distinct absorbance spectra, and their interconversion on this time scale can be monitoring using a UV-visible spectrophotometer for flash photolysis.) The second criterion is provided by the model's mean channel open time (˜3 s; \acute{α}⁻¹), i.e., the period during which the agonist remains locked. This period of ˜3 s provides a target upper limit of the photoswitch relaxation time. It is important to emphasize that these criteria derive from the assumption that the photoswitch cannot relax when the ligand is locked at the binding site. However, this assumption may not be correct. A highly exothermic cis-trans photoswitch isomerization may cause the receptor channel to close on a time scale faster than the intrinsic ˜3 s. Reciprocally, it is possible that the receptor might perturb the photoswitch relaxation kinetics. The occurrence of this latter possibility would likely be manifest as a reduced thermal isomerization rate of the photoswitch. In the event of such a distortion of receptor or photoswitch relaxation kinetics, we would retune the intrinsic photoswitch lifetime to compensate. The above considerations are based on the Chang & Weiss (1999) analysis of oocyte-expressed GABA_{C} receptors, the relaxation times of which are ˜5-10 times longer than those of native retinal GABA_{C} receptors (Qian & Ripps, 1999). The oocyte system will be a focus of initial electrophysiological testing (see Aim 2), however the performance of NNP assemblies with native retinal receptors may be re-assessed. Importantly, a fast-relaxing, “retinal GABA_{C}-tuned” device will likely be capable of eliciting measurable responses in slowly-relaxing oocyte-expressed GABA_{C} receptors, as bright light flashes can be used to drive the photoisomerization, and membrane current as little as 1% of the GABA-elicited maximum can be distinguished from baseline noise. In addition, it is likely that the performance criterion for a given receptor preparation may undergo changes for several reasons. One of these relates to the fact that GABA_{C} activation requires ligand binding at >2 of the receptor's five binding sites (Amin & Weiss, 1996; Karlin, 2002). If the NNP under investigation is monovalent, i.e., if a given photoswitch molecule regulates a single effector moiety (see, however, FIGS. 20-21), and assuming a 1-s lifetime of the photoactivated state, the requirement for temporally well-overlapping occupation of multiple ligand-binding sites on a given receptor translates to a requirement for photoactivating isomerizations of multiple NNPs on the receptor within a period short by comparison with 1 s. Assuming the objective of NNP function at bright but conventionally encountered levels of ambient light (at wavelengths absorbed by the photoswitch), it may become important to tune the photoswitch lifetime to significantly longer values, thereby sacrificing some temporal resolution of NNP function to assure multi-site ligand occupancy. Yet a further consideration is the relationship of photoswitch relaxation time to the period after photoisomerization that is required for diffusion of the effector to the receptor's ligand-binding site. This consideration is most applicable to the length of the tethering chain which may range up to ˜216 Å. The mean time T for diffusion of a molecule from the surface of a sphere of radius L to a target of radius b in the center is given by T=L3/3 Db (Berg & von Hippel, 1985). For consideration of this relation, we shall take L=216 Å as the chain length, b=10 Å as the radius of the ligand-binding site, and D=1×10⁻⁶ cm² s⁻¹ as the diffusion coefficient. The chosen value of the diffusion coefficient is appropriate for a small protein like lysozyme (MW 14,000) in water. Although a small molecule like sucrose (D=5×10⁻⁶ cm² s⁻¹ in water) might be viewed as a more appropriate reference due to its near-identity in molecular weight with the anticipate photoswitch effector couples, the value of 1×10⁻⁶ cm² s⁻¹ seems appropriate because of the expected tortuosity/viscosity of the extracellular space at the cell surface membrane, which typically reduces diffusion coefficients by 1.5-2.5 fold from their value in water (Nicholson & Sykova, 1998). With these values of L, D and b, the diffusion time T is equal to 34 μs, a period tiny by comparison with the targeted 30-ms lower limit of photoswitch relaxation time. As the diffusion coefficient grows approximately with the cube root of molecular weight, one would predict that the diffusion coefficient for PEG 3400 would have only an ˜2-fold effect on the above value of T.

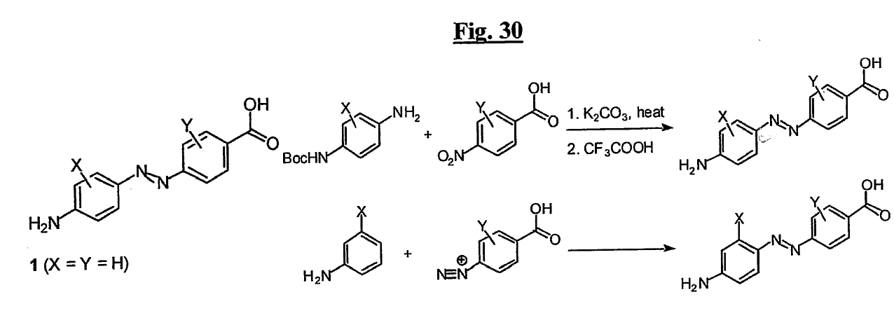

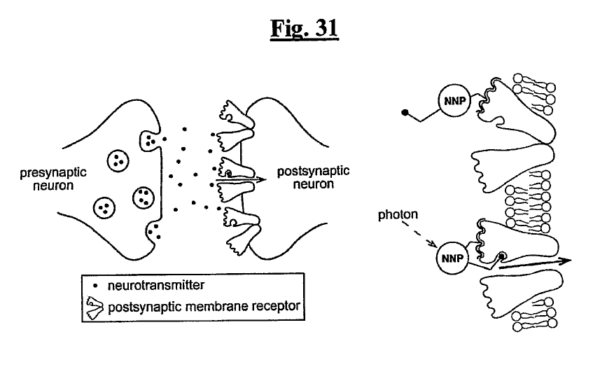

Primary Targets: Push-pull azobenzenes. Both relaxation time and isomerization wavelength in azobenzenes can be tuned through appropriate choice of substituents. Notable are “push-pull” azobenzenes, where an electron donor substituent on one ring is paired with an electron acceptor substituent on the other (Ross & Blanc, 1971; Kobayashi et al., 1987). Tuning is accomplished by varying the strength of the donor [e.g., CH₃<OCH₃<N(CH₃)₂], the strength of the acceptor (e.g., COOH<SO₂OH<NO₂), and their positions on the rings. (FIG. 30). Importantly, substituent combinations that lead to cis-trans relaxation rates in the target range typically shift the trans-cis excitation wavelength into the visible region due to the extended π-conjugation. Push-pull azobenzenes can be prepared by one of three routes: condensation of a nitroso compound with an aniline (cf. Ulysse & Chmielewski, 1994; Park & Standaert, 1999), condensation of a nitro compound with an aniline (FIG. 30, upper route), or coupling of a diazonium salt with an aniline or phenol (FIG. 30, lower route). Published spectral data and isomerization rates provide examples of compounds with visible-light absorbances and isomerization rates that bracket the target range. For example, 4-amino-4′-carboxyazobenzene (FIG. 30, compound 1), λₘₐₓ (trans) is 420 nm, and the time constant for cis-trans isomerization is 3 min. in DMSO (Wachtveitl et al., 1997). For 4-dimethylamino-4′-sulfoazobenzene, which has a more powerful donor-acceptor pair, λₘₐₓ is 480 nm (Oakes & Gratton, 1998), and the lifetime in water is 6.6 s at 25° C. (Asano & Okada, 1984). Use of an even more powerful 4-diethylamino-4′-nitro pair affords λₘₐₓ of 512 nm and a lifetime of 2.2 ms in DMSO. The same compound has a λₘₐₓ of 493 nm and a lifetime of 1.0 s in chloroform (Schanze et al., 1983). As this last example illustrates, thermal isomerization rates are highly sensitive to solvent, with polar solvents accelerating the process, and it is not yet clear which solvent will best model the micro-environment of the NNP photoswitch. Thus, we anticipate that identification of the appropriate donor/acceptor combination will require considerable effort in synthesis and empirical testing.

Alternative targets: While azobenzenes are the primary choice for the second-generation photoswitch, brief mention of other alternatives is appropriate. One potential class of targets are the imine (Schiff base) analogs of azobenzene, in which one N of the azo linkage is replaced with a CH. These are photoisomerizable, isosteric with azobenzene, and can exhibit thermal cis-trans relaxation times of about 1 s, even without push-pull substituents (Wettermark & Dogliotti, 1964; Anderson & Wettermark, 1965; Wettermark et al., 1965; Gorner & Fisher, 1991). Several other photoisomerizable organic structures have been closely investigated as switch nuclei. However, none are likely to be suitable because they have either or both of two problems: the need for UV photoactivation [spiropyrans (Hobley et al., 2003); spirooxazines (Metelitsa et al., 2002); naphthopyrans (Jockush et al., 2002; Gabbutt et al., 2005)] or thermal relaxation times well outside the target range [spiropyrans (Gorner, 2001); diarylethylenes and fulgides (Kobatake & Irie, 2003); thioindigos (Rosengaus & Willner, 1995; Fyles & Zeng, 1998); and hemithioindigos (Steinle & Rueck-Braun, 2003; Lougheed et al., 2004)]. Extended-lifetime core/shell nanocrystals. CdSe nanocrystals possess a large dipole moment (up to ˜60 Debye) that is believed to reflect the electrical polarization of interatomic bonds in the CdSe wurtzite crystal structure (Shim & Guyot-Sionnest, 1999). Photogeneration of an electron-hole pair significantly reduces this dipole moment, and in CdSe core and core/shell nanocrystals of ordinary composition, recombination of the electron-hole pair returns the nanocrystal's electronic structure to the pre-illumination state on a time scale of ˜10 ns (Javier et al., 2003). By analogy with a concept considered by Schmidt & Leach (2003) in which nanocrystals positioned at the membrane of nerve axons could be used to initiate action potentials, extension of the electron-hole lifetime to the μs range or greater could permit use of the photo-induced dipole perturbation as a photoswitch. If the above strategies to obtain an organic photoswitch that absorbs efficiently at visible wavelengths and spontaneously relaxes on the needed time scale are not acceptable using core/shell nanocrystals as the photoswitch component may be. This specifically involves engineering the core and shell bandgaps of CdSe/ZnSe nanocrystals to achieve a type-II offset of the valence and conduction bands, and (at Vanderbilt Univ.) pilot opto-electronic testing of the preparations to evaluate their potential suitability as a photoswitch component.

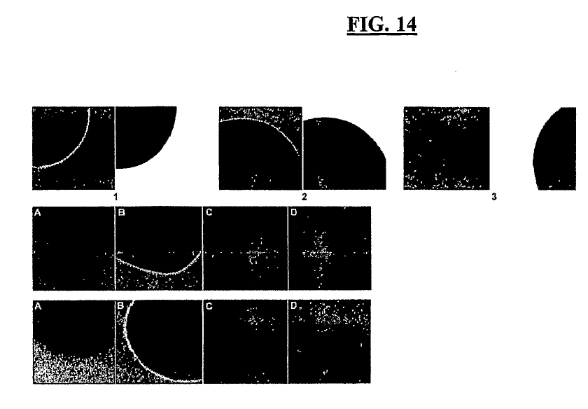

Preparation/testing of platform assemblies: The modular design of the NNP will allow assembly using conventional peptide coupling chemistry to join the effector/photoswitch/linker to a defined position on a photoaffinity-probe-derivatized anchor. Fully assembled candidate NNPs (i.e., structures in which the effector/photoswitch and PEG linker of a given test length joined to a defined amino acid position of the peptide anchor) may be used with isolated GABA_{C} extracellular domain and with GABA_{C}-expressing cells in biophysical/electrophysiological experiments (FIG. 14) to achieve transient, visible-light-stimulated GABA_{C} channel gating. The following considerations will be important to optimizing the assembly with respect to physiological performance. Short-wavelength photolability of muscimol: Muscimol is photolabile at wavelengths near 254 nm and in fact can act as a photoaffinity label at this wavelength (Cavalla & Neff, 1985). Many of the photoaffinity probes noted above, while anticipated to be workable for mapping the site of GABA_{C} attachment of a given peptide ligand and for determining silent modes of the peptide attachment, require activation with similar wavelengths and are likely to be unworkable for use as the covalent binding component in full NNP structures that employ muscimol as the effector moiety. For use in such muscimol-based, fully assembled structures, the use of photoaffinity probes such as benzophenones (Dorman & Prestwich, 1994) are activatable with light of 350-360 nm, where muscimol has negligible absorbance. Energetics of photoswitch cis vs. trans states: One of the design criteria discussed above is the use of cis-permissive azobenzenes, a choice dictated by the much larger thermodynamic stability of the trans isomer. Where the exponential lifetime of the thermal relaxation from cis to trans is on the order of 1 s, as is anticipated with push-pull azobenzenes, complete relaxation will occur in a few seconds in darkness, and a trans-permissive device would remain perpetually activated. It is of interest to consider how push-pull substitution affects this equilibrium, in conjunction with the binding of the NNP effector at the ligand-binding site. In azobenzene itself, the trans form is more stable by 49 kJ/mol (Dias et al., 1992). While corresponding data are not available for the fast-relaxing push-pull azobenzenes, the energy difference between the cis and trans forms of these compounds should be even greater due to the highly favorable conjugation of the push-pull groups in the trans isomer, which is disrupted in the cis isomer. We can conservatively retain the value of 49 kJ/mol as the energy difference between the cis and trans states. Even with a high-affinity effector like GABA (K_{D}˜1 μM, corresponding with a 34 kJ/mol binding energy; ΔG°=−RT [ln(K_{D}/(1 M)]}, the ligand-binding energy is still far lower than the cis-trans energy difference for the push-pull azobenzene. Thus, the thermodynamic preference for trans is expected to be 15 kJ/mol (49 kJ/mol-34 kJ/mol); the trans form is still favored by a factor of 400, and the thermal occupancy of the permissive, cis form is only 1/400.

Signal transmission at chemical synapses in the nervous system involves the action of receptor proteins at the postsynaptic membrane that respond to neurotransmitter released by the presynaptic neuron. Ligand-gated ion channels (LGICS) represent a major group of postsynaptic membrane receptors. LGIC receptors, which include GABA_{A}, GABA_{C}, glycine, serotonin and nicotinic acetylcholine receptors, exhibit a common overall structure consisting of five noncovalently assembled subunits. The ligand-binding sites of LGICs are located at junctions of the extracellular domains of adjacent subunits, and the subunits exhibit significant amino acid sequence homology. Although crystal structures are not yet available for any LGIC, the recent determination of the crystal structure of acetylcholine binding protein (a glial protein of the snail) (Brejc et al., 2001, Sixma & Smit, 2003) has afforded relatively detailed homology-based modeling of LGIC structure (Ernst et al., 2003). GABA is the major inhibitory neurotransmitter in the brain, and GABA_{A} receptors are widely distributed in the CNS. In addition to GABA binding sites, the GABA_{A} receptor exhibits modulatory sites sensitive to benzodiazepines, barbiturates and neurosteroids (Johnston, 1996), and the regulation of GABA_{A} activity by drugs targeting these sites is a major focus of psychiatric therapies.

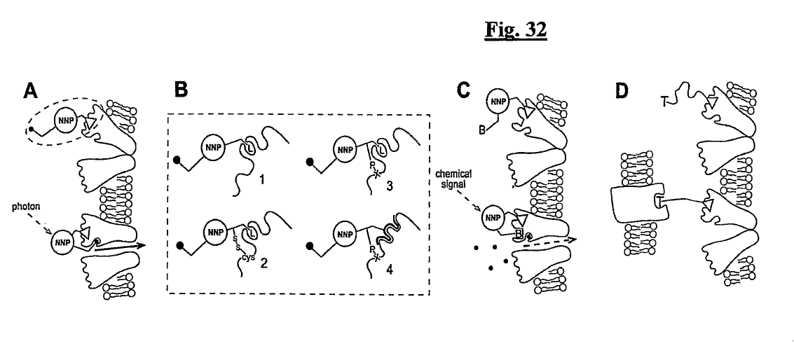

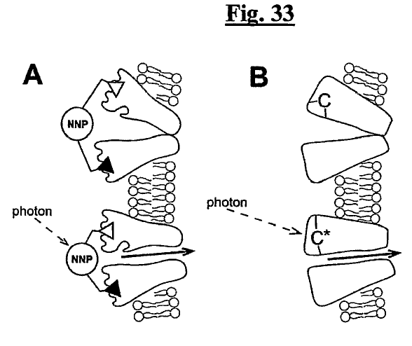

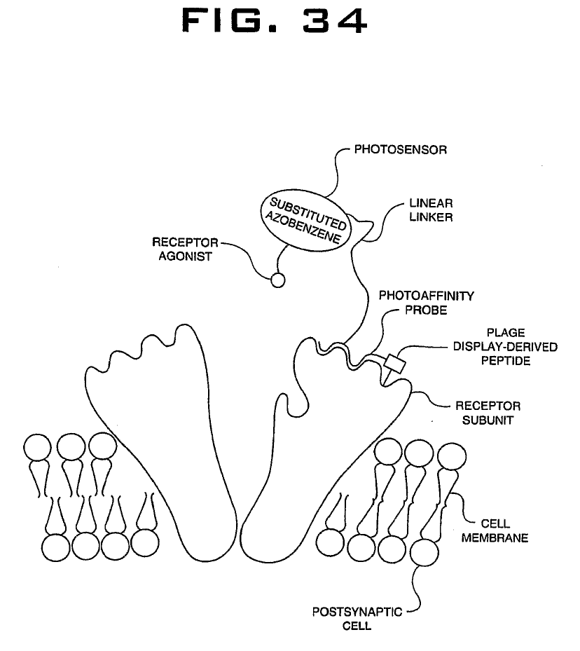

The objective of the procedures relating to the GABAa receptor referred to above is further described by FIGS. 31 and 32. FIG. 31 considers a molecular device (“nanoscale neuromodulating platform”, or NNP) proposed in that application as a therapy in retinal degenerative disease. The left-hand diagram of FIG. 31 describes signal transmission at a normally functioning chemical synapse. Here the postsynaptic membrane receptor is a (hypothetical) LGIC consisting of two subunits and a single ligand-binding site. Neurotransmitter (filled circles) released from the presynaptic neuron in response to stimulation diffuses across the synaptic cleft and binds to the postsynaptic receptors. The resulting activation of these receptor proteins opens transmembrane ion channels (inward-pointing arrow), thereby generating an electrical signal in the postsynaptic neuron. The right-hand diagram describes operation of the NNP envisioned for development. The diagram specifically considers the case of photoreceptor degenerative disease (e.g., age-related macular degeneration, in which retinal neurons postsynaptic to the degenerated rod and cone photoreceptors are believed in certain cases to remain potentially capable of function), and envisions the restoration of light-stimulated signaling in post-photoreceptor neurons by NNPs introduced into the diseased retina. The illustrated NNP consists of a neurotransmitter or analog (small filled circle; “effector” component) tethered to a chemical structure (circle labeled NNP) that incorporates a molecular photoswitch, and an anchoring moiety (thick wavy line) that attaches the introduced NNP at the extracellular face of postsynaptic receptors of specific post-photoreceptor neurons remaining healthy in the diseased retina. Photon absorption produces a transient conformational change in a linker arm that moves the effector to the receptor's ligand-binding site and thereby transiently activates the receptor, i.e., opens the receptor's ion channel. The NNP's anchoring moiety is a phage-display-derived peptide that noncovalently attaches the NNP to the postsynaptic receptor. As a self-contained photosensor with localized stimulating activity, the NNP would achieve the microspecific functionality required for meaningful visual signal initiation.

In FIG. 32A the anchoring portion of a representative functionalizing structure (here, the photosensitive NNP of FIG. 31) and the site of its covalent attachment to the GABA_{A} subunit are together symbolized by the open triangle. The FIG. 32B diagrams show in expanded view the region enclosed by the dashed oval in A and illustrate several attachment strategies. In strategy 1, a prototype approach not involving covalent attachment to the receptor, a genetically engineered amino acid sequence contains, as a recognition element, the inserted sequence of a binding protein with high affinity for its ligand, and (ii) a tethered form of this ligand (L) as part of the functionalizing structure. Immediate candidates for testing this strategy are FKBP (Standaert et al., 1990), a 107-amino acid binding protein that binds its FK506 ligand with known nanomolar affinity; and dihydrofolate reductase, a protein that has similarly high affinity for its inhibitory ligand, methotrexate (Kopytek et al., 2000). Additional strategies for insertion of a recognition element within a target protein have been described (e.g., Adams et al., 2002). Strategy 2 combines binding protein insertion with covalent anchoring of the functionalizing structure at a cysteine residue introduced by site-directed mutagenesis at a position neighboring the inserted binding protein. Here the functionalizing structure will be designed to incorporate a thiol-reactive moiety whose steric properties (e.g., length of an alkyl chain linking this moiety to the remainder of the structure) will favor bond formation specifically with the thiol group of the introduced cysteine. The rationale for this approach is that the high specificity of the functionalizing structure for the receptor's inserted binding protein will diminish nonspecific attachment to undesired cysteines and other thiol-containing molecules expected to be present on the surface of the cell expressing the target receptor. Strategies 3 and 4, conceptually similar to strategy 2, combine photoaffinity labeling with noncovalent attachment via either ligand-binding protein (3) or a phage-derived binding peptide (4; cf. FIG. 31). Here the functionalizing structure incorporates a tethered photoaffinity reagent P (aryl azide; e.g., Turek et al., 2002) whose steric position favors covalent linkage to a desired amino acid X of the receptor subunit upon UV illumination. A specific advantage of strategy 4 is its use of the native receptor subunit, a factor facilitating applications to LGICs of native CNS tissue. Critical to strategies 1-3 will be the identification of sites within the subunit's extracellular domain that afford expression/function of the desired sequence insertion/substitution while preserving physiological function of the receptor. Determining these permissive attachment sites involves homology-based and computational modeling using available sequence, structural and biochemical data (e.g., Brejc et al., 2001; Teissere & Czajkowski, 2001; Bera et al., 2002; Chang & Weiss, 2002; Ernst et al., 2003; Binkowski et al., 2003), and the testing of constructed receptors and anchoring moieties in biophysical, pharmacological and electrophysiological experiments.

Illustrated in FIG. 32C functionalization of the GABA_{A} receptor with a structure that contains a tethered benzodiazepine derivative (B) as effector, and in controlled fashion interacts with the receptor's benzodiazepine modulatory site. By contrast with conventional therapies involving administration of a freely diffusing drug, the covalent attachment of this structure would afford specific and localized action by the effector. Furthermore, regulation of the presentation of this effector by an external signal acting on the structure's signal-responsive element (in FIG. 32C, an administered synthetic chemical designed to have activity only at the signal-responsive component) would render this benzodiazepine-based therapy externally controllable by a highly specific, i.e., otherwise innocuous, drug. Moreover, the binding affinity of a given effector B could be tuned for a given disease or receptor type by the length/hydrophobicity of the chain tethering B, affording a new dimension of efficacy to the design of GABA_{A}-targeted therapies. FIG. 32D shows another potential application of receptor functionalization, that of interfacing the receptor with an introduced biological target or prosthetic device (e.g., a transplanted differentiated neuron or stem cell, or a neurotransmitter-releasing microfluidic system; Peterman et al., 2003) whose function requires an intimate association with the receptor. Here, the receptor would be functionalized with a (non-regulated) structure terminated by a molecular component (T) designed to have high affinity for a molecular component of the partner cell/device (cf. Movileanu et al., 2000). Upon introduction of the partner (in FIG. 32D, a transplanted cell with a known surface binding protein) to the LGIC receptor-containing tissue, T's binding to its target would tether the partner, thereby promoting its intended physiological interaction with the postsynaptic receptor.

In FIG. 33A a native LGIC functionalized with an introduced light-responsive structure (NNP) whose regulation of the receptor is mediated entirely through its covalent interactions with specific amino acid residues (open and filled triangles), i.e., whose operation does not require tethered forms of an activating receptor ligand or modulator. Panel 33B shows a fully synthetic light-sensitive protein whose synthesis within the neuron would be achieved by targeted gene therapy, and which responds to light (photic activation of a chromophore C akin to those of naturally occurring photoproteins) with a conformational change that opens an ion channel.

Initial constructs in model cells (Xenopus oocytes and HEK cells) are used to synthesize initial FK506-derivatized and aryl azide-(photoaffinity label-) containing compounds as test structures for subunit functionalization; and, through pharmacological/electrophysiological testing (e.g., Vu et al., 2004), to determine GABA_{A} activity in the transfected cells in the absence vs. presence of the functionalizing structure. Site-directed cysteine substitution in GABA_{A} subunits can determination intermolecular distances by fluorescence resonance energy transfer (FRET), computational molecular dynamics, and high-throughput assays for drug-receptor interactions.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents

What is claimed is:

1. A nanoscale neuromodulator platform apparatus for activating membrane receptors of a postsynaptic neuron in response to light, said apparatus comprising:
an effector;
a photoswitch;
said photoswitch having a first configuration and second configuration, said first configuration being adapted to operatively approximate said effector with a postsynaptic receptor such that the receptor is activated;
said second configuration maintaining said effector remote from said operative approximation with the postsynaptic receptor such that the receptor remains unactivated;
said photoswitch being mediated between said first configuration and said second configuration by exposure to a preconfigured range of electromagnetic radiation;
an anchor, said anchor being adapted to attach the apparatus to a native postsynaptic receptor area; and
a linker between said effector, said photoswitch and said anchor, said linker maintaining said effector within a range of the receptor sufficient for said effector to operatively approximate with the receptor when said photoswitch is in said first configuration.

2. The apparatus of claim 1 wherein said linker is a PEG chain.

3. The apparatus of claim 1 wherein said effector incorporates azobenzene.

4. The apparatus of claim 1 wherein a dynamic range of said effector combination is one order of magnitude.

5. The apparatus of claim 1 wherein said effector is an agonist.

6. The apparatus of claim 5 wherein said agonist is a muscimol derivative.

7. The apparatus of claim 1 wherein said receptor is a GABA_{C} receptor.

8. The apparatus of claim 1 further comprising a second effector.

9. The apparatus of claim 1 wherein said anchor includes covalent attachment to the receptor that preserves normal receptor function.

10. The apparatus of claim 1 wherein said receptor is a ligand-gated ion channel.

11. The apparatus of claim 1 wherein said effector is a neurotransmitter derivative.

12. The apparatus of claim 1 wherein said effector is a modulator of the receptor.

13. The apparatus of claim 1 wherein said mediation of said photoswitch is transient.

14. The apparatus of claim 1 wherein said photoswitch spontaneously reverts to said second configuration after being put in said first configuration by said exposure to said preconfigured range of electromagnetic radiation.

15. The apparatus of claim 1 wherein said preconfigured range of electromagnetic radiation is visible light.

16. The apparatus of claim 1 wherein said effector is an antagonist.

17. The apparatus of claim 1 wherein said agonist is a neurotransmitter analogue.

18. The apparatus of claim 1 wherein said anchor incorporates peptides derived from phage display screening.

19. The apparatus of claim 1 wherein said anchor incorporates non-covalent binding of the apparatus to the receptor.

20. The apparatus of claim 1 wherein said anchor includes a photoaffinity probe.

21. The apparatus of claim 1 wherein said receptor is a metabotropic receptor.
